(19) United States
(12) Patent Application Publication
(10) Pub. No.: US 2021/0315816 A1
(43) Pub. Date: Oct. 14, 2021

(54) ANTIMICROBIAL COMPOSITIONS AND METHODS

(71) Applicant: BOARD OF REGENTS OF THE UNIVERSITY OF NEBRASKA, Lincoln, NE (US)

(72) Inventors: Tammy Kielian, La Vista, NE (US); Tatiana Bronich, Elkhorn, NE (US); Kelsey Yamada, Omaha, NE (US); Xinyuan Xi, Omaha, NE (US)

(21) Appl. No.: 17/261,074

(22) PCT Filed: Sep. 12, 2019
(86) PCT No.: PCT/US2019/050780

§ 371 Date: Jan. 18, 2021

Related U.S. Application Data
(60) Provisional application No. 62/730,229, filed on Sep. 12, 2018.

(51) Int. Cl. A61K 9/107; A61K 31/365; A61K 47/66; A61K 45/06; A61P 31/04

(52) U.S. Cl. CPC A61K 9/1075; A61K 31/365; B82Y 5/00; A61K 45/06; A61P 31/04; A61K 47/66

(57) ABSTRACT

The present invention provides compositions and methods for the inhibition of a bacterial infection. More specifically, the present invention relates to compositions and methods for the inhibition and/or treatment of a bacterial infection, particularly an infection characterized by a biofilm. In a particular embodiment, the micelle comprises at least one block copolymer comprising an ionically charged polymeric segment and a non-ionically charged polymeric segment, wherein the ionically charged polymeric segment is grafted with hydrophobic moieties

This application claims priority under 35 U.S.C. § 119(e) to U.S. Provisional Patent Application No. 62/730,229, filed Sep. 12, 2018. The foregoing application is incorporated by reference herein.

This invention was made with government support under Grant No. P01 AI083211 awarded by the National Institutes of Health/National Institute of Allergy and Infectious Disease. The government has certain rights in the invention.

### FIELD OF THE INVENTION

The present invention relates generally to antimicrobials. More specifically, the present invention relates to compositions and methods for the inhibition and/or treatment of a bacterial infection, particularly an infection characterized by a biofilm.

### BACKGROUND OF THE INVENTION

Staphylococcus aureus is a leading cause of community-acquired and nosocomial infections that are associated with significant morbidity and mortality rates ranging from 15-60% (Fowler, et al. (2003) Arch Intern Med., 163:2066-2072; Morgenstern, et al. (2016) PLoS One 11:e0148437; Naber, C. K. (2009) Clin Infect Dis., 48 (Suppl 4):S231-237; Walls, et al. (2008) Bone Joint J., 90-B:292-298; Self, et al. (2016) Clin. Infect. Dis., 63:300-309; Tande, et al. (2014) MBio 5:e01910-01914; Tande, et al. (2014) Clin. Microbiol. Rev., 27:302-345; Bishara, et al. (2012) Int. J. Infect. Dis., 16:e457-463). Currently, methicillin-resistant S. aureus (MRSA) accounts for greater than 50% of all nosocomial isolates and causes an estimated 18,000 deaths and 80,000 invasive infections in the United States annually (Fowler, et al. (2003) Arch Intern Med., 163:2066-2072; Morgenstern, et al. (2016) PLoS One 11:e0148437). The ability to form a biofilm allows S. aureus to colonize biotic surfaces as well as medical devices, including prosthesis, indwelling catheters, and stents (Naber, C. K. (2009) Clin Infect Dis., 48 (Suppl 4):S231-237; Guggenbichler, et al. (2011) GMS Krankenhhyg Interdiszip 6:Doc18; Klein, et al. (2007) Emerg. Infect. Dis., 13:1840-1846). Many bacterial cells within the biofilm are metabolically dormant, which contributes to their antibiotic resistance, making these infections difficult to eradicate with antibiotic therapy alone (Tande, et al. (2014) Clin. Microbiol. Rev., 27:302-345; Moormeier, et al. (2017) Mol. Microbiol., 104:365-376). One example of biofilm growth that is not readily cleared by the immune system or antibiotics is prosthetic joint infection (PJI).

The frequency of orthopedic procedures, such as total knee and total hip arthroplasty (TKA and THA, respectively), continues to increase and is predicted to reach an annual rate in the United States of 572,000 and 3.48 million, respectively, by 2030 (Kurtz, et al. (2007) J. Bone Joint Surg. Am., 89:780-785; Kurtz, et al. (2007) J. Bone Joint Surg. Am., 89 (Suppl 3):144-151). The estimated annual incidence of PJI in the United States is 2.18% for all THAs and TKAs. However, the infection rate following revision surgery is even higher (i.e. 3.2-5.6% for both THA and TKAs). For PJIs, the current standard-of-care is to remove the infected hardware, fill the joint with an antibiotic-impregnated spacer, followed by a second surgery to replace the prosthesis (Tande, et al. (2014) Clin. Microbiol. Rev., 27:302-345; Moran, et al. (2010) J. Antimicrob. Chemo., 65(Suppl 3):iii45-54; Pulido, et al. (2008) Clin. Orthop. Relat. Res., 466(7):1710-5). This process can persist for months, depending on the time required to clear the infection, resulting in significant morbidity and medical expenses. This demonstrates the importance of developing novel therapeutic approaches to treat biofilm-associated infections without requiring multiple surgical interventions (Tande, et al. (2014) MBio 5:e01910-01914; Tande, et al. (2014) Clin. Microbiol. Rev., 27:302-345; Teterycz, et al. (2010) Intl. J. Inf. Dis., 14:e913-e918; Moran, et al. (2010) J. Antimicrob. Chemother., 65 (Suppl 3):iii45-54; Pulido, et al. (2008) Clin. Orthop. Relat. Res., 466:1710-1715). Improved therapeutics and methods for treating bacterial infections are needed.

### SUMMARY OF THE INVENTION

In accordance with the instant invention, therapeutic polymer micelles or nanoparticles are provided. In a particular embodiment, the micelle comprises at least one block copolymer comprising an ionically charged polymeric segment and a non-ionically charged polymeric segment, wherein the ionically charged polymeric segment is grafted with hydrophobic moieties (e.g., a hydrophobic amino acid). The hydrophobized ionically charged polymeric segment forms the core of the micelle and the non-ionically charged polymeric segment is hydrophilic and forms the shell of the micelle. For stability, the core of the micelle may be cross-linked. In certain embodiment, the micelle comprises a leukocyte specific targeting moiety, wherein the targeting moiety is linked to the non-ionically charged polymeric segment. In certain embodiments, the targeting moiety specifically targets or binds to monocytes and/or macrophages. In certain embodiments, the targeting moiety is tuftsin. In certain embodiments, the micelle comprises an inhibitor of oxidative phosphorylation (e.g., within the core of the micelle), such as an oligomycin. Compositions comprising the micelles of the instant invention are also provided.

In accordance with another aspect of the instant invention, methods for treating, inhibiting, and/or preventing a bacterial infection in a subject are provided. In certain embodiments, the method comprises administering to the subject at least one micelle of the instant invention (optionally in a composition with a pharmaceutically acceptable carrier). The methods may further comprise the administration of other therapeutic methods or compositions to the subject. In certain embodiments, the method further comprises administering an antibiotic and/or antibacterial drug to the subject (e.g., systemically).

### BRIEF DESCRIPTIONS OF THE DRAWING

FIGS. 1A-1B show that S. aureus biofilm infection promotes a shift towards OxPhos metabolism in monocytes. Monocytes associated with tissues surrounding the knee joint of mice with S. aureus-infected (I) or sterile (S) orthopedic implants were stained with (A) the bi-potential dye JC-1 or (B) 2-NBDG at the indicated time points (days 1-7) as measures of OxPhos and glycolysis, respectively, and analyzed by flow cytometry. FIG. 1A: OxPhos was calculated as the ratio of green:red monocytes (CD11b^{high}Ly6G⁻Ly6C⁺F4/80⁻) following JC-1 staining and is reported as a percentage relative to animals receiving sterile implants. FIG. 1B: Glycolytic activity is reported as the percentage of monocytes that were 2-NBDG+ relative to mice with sterile implants. Data are presented as the mean±SD (n=10 animals/group) combined from two independent experiments (*, p<0.05; **,p<0.01; ***,p<0.001; ****,p<0.0001; Student's t-test).

FIGS. 2A-2B show that inhibition of OxPhos by oligomycin promotes macrophage pro-inflammatory activity. Bone marrow-derived macrophages were plated overnight and the following day cells were pre-treated with IL-4 (10 ng/mL) for 1 hour followed by various concentrations of oligomycin for 24 hours, whereupon TNF-α (FIG. 2A) and arginase (FIG. 2B) expression was determined by ELISA and enzymatic assay, respectively. Data are presented as the mean±SD combined from two independent experiments (n=6 biological replicates) (*, p<0.05; **, p<0.01; ***, p<0.001; Student's t-test).

FIGS. 3A-3E show that oligomycin nanoparticles shift macrophage metabolism towards glycolytic pathways. Bone marrow-derived macrophages were treated with IL-4 (10 ng/mL); peptidoglycan (PGN)+IFN-γ (10 μg/mL and 10 ng/mL, respectively); or Cy5/Tuftsin/Oligomycin (CTO) nanoparticles (1 μg/mL) for 24 hours, whereupon bio-energetic profile assays were performed to assess mitochondrial function (FIG. 3A) and glycolytic rates (FIG. 3B). Basal (FIG. 3C) and maximal (FIG. 3D) respiratory, and glycolytic (FIG. 3E) rates were calculated using well-defined algorithms (*,p<0.05; **,p<0.01; ****,p<0.0001). ns, not significant.

FIGS. 4A-4G show the preferential nanoparticle uptake by monocytes/macrophages during PJI. C57BL/6NCrl mice received a single intra-articular injection of Cy5 (C) or Cy5/Tuftsin (CT) nanoparticles (10 μg) at day 7 post-infection and analyzed on three consecutive days (FIG. 4A). IVIS imaging of labeled nanoparticles in the infected joint at day 3 (FIG. 4B) with representative flow plots (FIG. 4C). Left panel shows all CD45⁺ cells and the right panel depicts P1 gated cells, where Cy5⁺ monocytes (CD45⁺ Ly6G⁻Ly6C⁺CD11b⁺) are highlighted in black. Mice receiving C or CT nanoparticles were sacrificed at days 1-3 following nanoparticle treatment, whereupon Cy5⁺ monocytes were quantified by flow cytometry (FIG. 4D). Data is presented as the mean±SD (n=4-5 mice/time point). (*, p<0.05; ***, p<0.001; Student's t-test). FIG. 4E shoes the longevity of nanoparticle retention at the site of S. aureus biofilm infection. C57BL/6NCrl mice received a single intra-articular injection of Cy5 labeled nanoparticles at day 3 post-infection, whereupon the same animal was imaged over a two week period to demonstrate the extent of nanoparticle retention and distribution. Results are representative of five individual mice. FIG. 4F shows that oligomycin-containing nanoparticles affect S. aureus biofilm burdens 7 days post-infection. C57BL/6NCrl mice received a single intra-articular injection of C, CT, or CTO nanoparticles at day 7 post-infection, whereupon animals were sacrificed 3 or 7 days following nanoparticle treatment. Bacterial burdens were quantified from the surrounding soft tissue, knee, femur, and implant. Results are from one experiment (n=5 mice/group/time point); (*,p<0.05; **,p<0.01; ***, p<0.001). FIG. 4G provides a graph of oligomycin release from Cy5-tuftsin-oligomycin (CTO) nanoparticles determined using a PBS dialysis method with a 3.5 kDa membrane cutoff. The kinetics of oligomycin release was determined by HPLC and concentrations are expressed as a percentage of the total oligomycin available vs. time (in hours; h).

FIGS. 5A-5B show that oligomycin-containing nanoparticles induce a metabolic shift in biofilm-associated monocytes in vivo. C57BL/6NCrl mice received a single intra-articular injection of CT or CTO nanoparticles (10 μg) at day 7 post-infection (n=5 mice/treatment group). Mice were sacrificed 3 days following nanoparticle injection and monocytes (CD11b^{high}Ly6G⁻Ly6C⁺F4/80⁻) were recovered from infected tissues by FACS, whereupon intracellular metabolites were isolated for analysis by HPLC/MS/MS. FIG. 5A shows a principle component analysis (PCA) plot was generated using an algorithm with mean intensities and pareto scaling distribution. Ellipses represent a 95% confidence interval of the normal distribution for each cluster. FIG. 5B shows the heat-map for the top 25 metabolite differences in monocytes recovered from CTO and CT treated mice. The key indicates log₂-fold changes of normalized-mean peak intensities for metabolites in monocytes from CTO treated animals normalized to the CT nanoparticle treated group.

FIG. 6 shows that oligomycin-containing nanoparticles polarize monocytes towards a pro-inflammatory phenotype in vivo. C57BL/6NCrl mice received a single intra-articular injection of CT or CTO nanoparticles (10 μg) at day 7 post-infection (n=5 mice/treatment group). Mice were sacrificed 3 days following nanoparticle injection and monocytes (CD11b^{high}Ly6G⁻Ly6C⁺F4/80⁻) were sorted from infected tissues by FACS, whereupon RNA was immediately isolated for RT-qPCR. Gene expression levels in monocytes recovered from CTO-treated animals were calculated after normalizing signals to GAPDH and are presented as the fold-change relative to monocytes isolated from mice receiving CT (control) nanoparticles. Data is presented as the mean±SD (n=3 sets of monocytes) combined from three independent experiments.

FIGS. 7A-7C show that oligomycin-containing nanoparticles targeted to monocytes induce metabolomic and gene expression changes in myeloid-derived suppressor cells (MDSCs). C57BL/6NCrl mice received a single intra-articular injection of CT or CTO nanoparticles (10 μg) at day 7 post-infection. Mice were sacrificed 3 days following nanoparticle injection and MDSCs (CD11b^{high}Ly6G⁻Ly6C⁺F4/80⁻) were recovered from infected tissues by FACS, whereupon intracellular metabolites and RNA were isolated for analysis by HPLC/MS/MS and RT-qPCR, respectively. FIG. 7A provides a PCA plot generated using an algorithm with mean intensities and pareto scaling distribution. Ellipses represent a 95% confidence interval of the normal distribution for each cluster. FIG. 7B provides a heat-map depicting the top 25 metabolite differences in MDSCs recovered from CTO and CT treated mice. The key indicates log₂-fold changes of normalized-mean peak intensities for metabolites in MDSCs from CTO treated animals normalized to the CT nanoparticle treated group. FIG. 7C provides gene expression levels in MDSCs recovered from CTO-treated animals calculated after normalizing signals to GAPDH and presented as the fold-change relative to MDSCs isolated from mice receiving CT (control) nanoparticles. Results are combined from three independent experiments.

FIGS. 8A-8I shows that nanoparticle-mediated delivery of oligomycin to monocytes/macrophages reduces established biofilm burden. C57BL/6NCrl mice received a single intra-articular injection of C, CT, or CTO nanoparticles at day 7 post-infection, and were analyzed at 7, 14, 21, or 28 days following nanoparticle treatment (FIG. 8A). Bacterial burdens were quantified in the surrounding soft tissue (FIG. 8B), knee (FIG. 8C), femur (FIG. 8D), and implant (FIG. 8E), wherein the short lines and the dotted lines represent the mean and limit of detection (LOD), respectively. Infiltrating leukocytes were analyzed by flow cytometry and MDSCs (FIG. 8F), PMNs (FIG. 8G), monocytes (FIG. 8H), and macrophages (FIG. 8I) are reported as a percentage of live CD45⁺ leukocytes. Results are combined from three independent experiments (n=15 mice/group/time point); (*,p<0.05; **,p<0.01; ***,p<0.001; ****,p<0.0001; one-way ANOVA).

FIGS. 9A-9H shows that oligomycin-containing nanoparticles act synergistically with antibiotics to clear established S. aureus biofilm infection. C57BL/6NCrl mice received a single intra-articular injection of CT or CTO nanoparticles (10 μg) at day 7 post-infection. Seven days later, animals received daily i.p. injections of antibiotics (25 mg/kg/day rifampin and 5 mg/kg/day daptomycin) or vehicle for one week, whereupon mice were sacrificed at day 21 post-infection. Bacterial burdens were quantified from the surrounding soft tissue (FIG. 9A), knee (FIG. 9B), femur (FIG. 9C), and implant (FIG. 9D). Infiltrating leukocytes were analyzed by flow cytometry and MDSCs (FIG. 9E), PMNs (FIG. 9F), monocytes (FIG. 9G), and macrophages (FIG. 9H) are reported as a percentage of live CD45⁺ leukocytes. Results are combined from two independent experiments (n=10 mice/group/time point); (*, p<0.05; **, p<0.01; ***, p<0.001; ****, p<0.0001; one-way ANOVA). Dotted line represents the limit of detection (LOD).

FIGS. 10A-10I show that nanoparticle-mediated delivery of oligomycin 3 days post-infection to monocytes/macrophages reduces biofilm burden. C57BL/6NCrl mice received a single intra-articular injection of C, CT, or CTO nanoparticles at day 3 post-infection, and were analyzed at 7, 14, 21, or 28 days following nanoparticle treatment (FIG. 10A). Bacterial burdens were quantified in the surrounding soft tissue (FIG. 10B), knee (FIG. 10C), femur (FIG. 10D), and implant (FIG. 10E). Infiltrating leukocytes were analyzed by flow cytometry and MDSCs (FIG. 10F), PMNs (FIG. 10G), monocytes (FIG. 10H), and macrophages (FIG. 10I) are reported as a percentage of live CD45⁺ leukocytes. Results are combined from three independent experiments (n=15 mice/group/time point); (*, p<0.05; **, p<0.01; ***, p<0.001; ****, p<0.0001; one-way ANOVA).

FIG. 11 shows that oligomycin does not directly affect S. aureus biofilm bacterial burdens. C57BL/6NCrl mice received intra-articular injections of oligomycin at 7 day post-infection (100 ng), or two sequential doses at days 7 and 8 post-infection (50 ng/day), or PBS and were sacrificed at day 14 post-infection. Bacterial burdens were quantified from the surrounding soft tissue, knee, femur, and implant. Results are from one experiment (n=5 mice/group/time point); (*, p<0.05; one-way ANOVA). Additionally, S. aureus biofilm formation was assessed in 96-well plates±oligomycin treatment (bottom graphs). Oligomycin (10 μg/mL) was added to planktonic S. aureus at time 0 (left panel) or mature biofilms for 4 days (right panel) to determine effects on bacterial growth at the indicated time points following oligomycin treatment. Cultures were replenished daily with fresh medium containing oligomycin. Results are presented as Log₁₀ colony forming units (CFU) per well (mean±SD).

FIGS. 12A-12D show that nanoparticle-mediated delivery of oligomycin attenuates OxPhos metabolism in biofilm-associated monocytes. C57BL/6NCrl mice received a single intra-articular injection of free oligomycin only (Oligo; 100 ng), empty nanoparticles (CT), empty nanoparticles (CT)+free oligomycin (100 ng; not loaded), or oligomycin loaded nanoparticles (CTO) at day 7 post-infection and were analyzed at day 3 (FIG. 12A, 12B; 5 mice/group) or 7 (FIGS. 12C, 12D; 9-10 mice/group) after treatment. FIGS. 12A, 12C: OxPhos was calculated as the ratio of green:red monocytes (CD11b^{high}Ly6G⁻Ly6C⁺F4/80⁻) following JC-1 staining and glycolytic activity was calculated as the percentage of 2-NBDG+ monocytes. FIGS. 12B, 12D: Bacterial burdens were assessed in the surrounding soft tissue, knee, femur, and implant, where the dotted lines represent the limit of detection (LOD). All results are reported as the mean±SD (*, p<0.05; **, p<0.01; ***, p<0.001; ****, p<0.0001; One-way ANOVA).

### DETAILED DESCRIPTION OF THE INVENTION

Biofilm-associated prosthetic joint infections (PJIs) cause significant morbidity and economic burden due to their recalcitrance to immune-mediated clearance and antibiotic therapy. Many PJIs are caused by gram-positive pathogens, including S. aureus. S. aureus biofilm-associated monocytes are polarized to an anti-inflammatory state and the adoptive transfer of pro-inflammatory macrophages attenuated biofilm burden, highlighting the critical role of monocyte/macrophage polarization state in dictating biofilm persistence. The inflammatory phenotype of leukocytes is linked to their metabolic state. Here, it is shown that the anti-inflammatory properties of biofilm-associated monocytes are coupled with a bias favoring OxPhos and less aerobic glycolysis to facilitate their anti-inflammatory activity and biofilm persistence. To shift monocyte metabolism in vivo and reprogram cells to a pro-inflammatory state, a novel nanoparticle approach was used to deliver the OxPhos inhibitor oligomycin to monocytes. Using a mouse model of S. aureus PJI, >90% of nanoparticle uptake was specific for monocytes, which significantly reduced S. aureus biofilm burden by altering metabolism and promoting pro-inflammatory properties of infiltrating monocytes as reveled by metabolomic and RT-qPCR analysis, respectively. Injection of oligomycin alone had no effect on monocyte metabolism or biofilm burdens, establishing that intracellular delivery of oligomycin is required to reprogram monocyte metabolic activity and that oligomycin lacks antibacterial activity against S. aureus biofilms. Remarkably, monocyte metabolic reprogramming with oligomycin nanoparticles was effective at clearing established biofilms, particularly in combination with systemic antibiotics. These findings indicate that metabolic reprogramming of biofilm-associated monocytes represents a novel therapeutic approach for bacterial infections such as PJIs.

S. aureus is a leading cause of PJI that is characterized by biofilm formation (Tande, et al. (2014) Clin. Microbiol. Rev., 27:302-345), and with the increased prevalence of methicillin-resistant S. aureus (MRSA) this pathogen has become an even greater therapeutic challenge (Turner, et al. (2019) Nat. Rev. Microbiol., 17(4):203-18). Biofilm infections persist despite antibiotic therapy due, in part, to decreased metabolic activity of a subpopulation of biofilm-associated bacteria and evasion of the immune response (Tande, et al. (2014) Clin. Microbiol. Rev., 27:302-345; Moormeie, et al. (2017) Mol. Microbiol., 104(3):365-76; Conlon, et al. (2016) Nat. Microbiol., 1:16051; Gries, et al. (2017) J. Am. Acad. Orthop. Surg., 25(Suppl 1):S20-S4; Balaban, et al. (2019) Nat. Rev. Microbiol., 17(7):441-8). S. aureus biofilms skew the host immune response towards an anti-inflammatory state (Thurlow, et al. (2011) J. Immunol., 186:6585-6596). This is evident by the polarization of anti-inflammatory monocytes/macrophages, abundance of MDSCs, and paucity of neutrophils and T cells (Hanke, et al. (2013) J. Immunol., 190:2159-2168; Hanke, et al. (2012) Front. Cell Infect. Microbiol., 2:62; Heim, et al. (2014) Methods Mol. Biol., 1106:183-191; Heim, et al. (2015) J. Leukoc. Biol., 98:1003-1013; Heim, et al. (2014) J. Immunol., 192:3778-3792). Two major MDSC subsets have been described, namely granulocytic- and monocytic MDSCs (G-MDSCs and M-MDSCs, respectively) that possess the capacity to differentiate into mature granulocytes or macrophages given the appropriate environmental cues (De Veirman, et al. (2014) Front. Oncol., 4:349; Dilek, et al. (2010) Curr. Opin. Organ Transplant 15:765-768; Gabrilovich, et al. (2009) Nat. Rev. Immunol., 9:162-174; Gabrilovich, et al. (2012) Nat. Rev. Immunol., 12:253-268; Bronte, et al. (2016) Nat. Comm., 7:12150). However, during chronic pathological conditions, such as cancer, infection, or autoimmune disorders, MDSCs are often arrested in an immature state where they can inhibit T cell activation and monocyte/macrophage pro-inflammatory properties (De Veirman, et al. (2014) Front. Oncol., 4:349; Dilek, et al. (2010) Curr. Opin. Organ Transplant 15:765-768; Gabrilovich, et al. (2009) Nat. Rev. Immunol., 9:162-174). G-MDSCs are integral to S. aureus biofilm establishment and persistence as well as polarizing infiltrating monocytes towards an anti-inflammatory phenotype (Heim, et al. (2015) J. Leukoc. Biol., 98:1003-1013; Heim, et al. (2014) J. Immunol., 192:3778-3792; Heim, et al. (2015) J. Immunol., 194:3861-3872). These effects are mediated, in part, by IL-10 production by MDSCs, which can block NF-κB activation in monocytes and macrophages and down-regulate cytokine expression (Heim, et al. (2015) J. Leukoc. Biol., 98:1003-1013; Heim, et al. (2014) J. Immunol., 192:3778-3792; Heim et al. (2018) J. Orthop. Res., 36(6):1605-1613). In addition, MDSC depletion augments monocyte pro-inflammatory activity, which translated into reduced biofilm burden (Heim, et al. (2015) J. Leukoc. Biol., 98:1003-1013; Heim, et al. (2014) J. Immunol., 192:3778-3792). Biofilm clearance is also facilitated by the adoptive transfer of pro-inflammatory macrophages at the site of biofilm infection (Hanke, et al. (2013) J. Immunol., 190:2159-2168). Therefore, the immune polarization state of monocytes and macrophages also plays a key role in dictating biofilm persistence and this is influenced, in part, by MDSC-derived factors.

The inflammatory phenotype of macrophages is intimately tied to their metabolic state. For example, anti-inflammatory macrophages rely primarily on oxidative phosphorylation (OxPhos) to drive their suppressive activity (Geeraerts, et al. (2017) Front. Immunol., 8:289; O'Neill, et al. (2016) J. Exp. Med., 213:15-23; Tavakoli, et al. (2013) J. Nucl. Med., 54:1661-1667; Torres, et al. (2016) Elife 5:e14354). However, upon exposure to pro-inflammatory stimuli, macrophages favor aerobic glycolysis (Geeraerts, et al. (2017) Front. Immunol., 8:289; O'Neill, et al. (2016) J. Exp. Med., 213:15-23; Tavakoli, et al. (2013) J. Nucl. Med., 54:1661-1667; Torres, et al. (2016) Elife 5:e14354; Galvan-Pena, et al. (2014) Front. Immunol., 5:420; Odegaard, et al. (2011) Annu. Rev. Pathol., 6:275-297). In vitro studies have shown that macrophage metabolic switches are facilitated by global changes in gene expression. For example, pro-inflammatory macrophages express the highly active PFK-2 isoform u-PFK2 (ubiquitous phosphofructokinase) and down-regulate TCA cycle enzymes, promoting intracellular glucose, succinate, and citrate accumulation (Geeraerts, et al. (2017) Front. Immunol., 8:289; O'Neill, et al. (2016) J. Exp. Med., 213:15-23). In contrast, anti-inflammatory macrophages express the less active PFK-2 isoform PFKB1 and upregulate CD36 to facilitate triglyceride uptake to fuel the TCA cycle (Geeraerts, et al. (2017) Front. Immunol., 8:289; O'Neill, et al. (2016) J. Exp. Med., 213:15-23; Feingold, et al. (2012) Biochem. Biophys. Res. Commun., 421:612-615; Feingold, et al. (2012) J. Leukoc. Biol., 92:829-839). In solid tumors, macrophages can be forced to undergo metabolic shifts through changes in oxygen, nutrient, and metabolite availability, which coincide with changes in inflammatory phenotype (Laoui, et al. (2014) Front Immunol., 5:489; Laoui, et al. (2014) Cancer Res 74:24-30; Van Overmeire, et al. (2016) Cancer Res., 76:35-42). Similarly, S. aureus biofilms generate nutrient, proton, and oxygen gradients with the potential to metabolically reprogram monocytes/macrophages and alter their inflammatory profile (Archer, et al. (2011) Virulence 2:445-459; Kiamco, et al. (2017) Appl. Environ. Microbiol., 83(6): e02783-16; Scherr, et al. (2015) MBio 6(4):e01021-15; Scherr, et al. (2014) Front. Immunol., 5:37; Stewart, et al. (2008) Nat. Rev. Microbiol., 6:199-210; Stewart, et al. (2016) NPJ Biofilms Microbiomes 2:16012). Of note, monocytes outnumber macrophages in a mouse PJI model and although monocytes are also polarized toward an anti-inflammatory state during S. aureus biofilm infection (Heim, et al. (2015) J. Leukoc. Biol., 98:1003-1013; Heim, et al. (2014) J. Immunol., 192:3778-3792; Heim, et al. (2015) J. Immunol., 194:3861-3872; Heim, et al. (2017) J. Orthop. Res., 36(6):1605-1613). However, it was not known whether these anti-inflammatory monocytes can be metabolically reprogrammed to promote their pro-inflammatory properties and biofilm clearance. Monocyte metabolism and inflammatory phenotypes exist within a spectrum and adapt in response to environmental stimuli (Martinez, et al. (2014) F1000prime Rep., 6:13; Nahrendorf, et al. (2016) Cir. Res., 119(3):414-7).

Herein, it is demonstrated that biofilm-associated monocytes are biased towards OxPhos compared to aerobic glycolysis, indicating that this is a key determinant to account for their anti-inflammatory properties. To further study this observation, novel cell-targeted nanoparticles containing the OxPhos inhibitor, oligomycin, were designed to re-program biofilm-associated monocytes to favor aerobic glycolysis and pro-inflammatory activity (Izquierdo, et al. (2015) J. Immunol., 195:2442-2451). Nanoparticles were conjugated with tuftsin, a tetrapeptide derived from the Fc domain of the IgG heavy chain, to target FcR-mediated uptake in monocytes (Jain, et al. (2012) Biomacromolecules 13:1074-1085; Jain, et al. (2015) Biomaterials 61:162-177). In vivo, Cy5-labeled oligomycin nanoparticles were preferentially internalized by monocytes, with minimal uptake by MDSCs or PMNs. Using a mouse model of S. aureus PJI, treatment of established biofilms with oligomycin nanoparticles shifted monocyte metabolism to a pro-inflammatory state concomitant with increased neutrophil and monocyte recruitment, resulting in significant reductions in S. aureus burden. These effects were not recapitulated with free oligomycin, establishing that intracellular delivery of oligomycin to monocytes is important and that oligomycin does not exert direct bactericidal activity against S. aureus. Remarkably, combined treatment with oligomycin nanoparticles and systemic antibiotics reduced the titer of established biofilms below the limit of detection, a finding that has never been achieved for a mature biofilm in vivo. Collectively, monocyte metabolic re-programming represents a novel therapeutic avenue for circumventing the two-stage revision protocol for patients with PJI by treating an infected implant in situ and alleviating a second surgery, which would represent a significant reduction in patient morbidity.

In accordance with the instant invention, targeted delivery vehicles are provided. Examples of delivery vehicles of the invention include, without limitation, nanoparticles, liposomes, and micelles. In certain embodiments, the delivery vehicle is a micelle (e.g., nanoscale micelle (e.g., up to about 1 μm in diameter)). In certain embodiments, the delivery vehicle comprises one or more polymers (e.g., a polymer micelle). Examples of micelles that can be used in the present invention are provided in U.S. Patent Application No. 2014/0039068, Desale et al. (J. Controlled Rel. (2015) 220(Pt B):651-9, and Kim et al. (J. Drug Target (2013) 21: 981-993), each of which are incorporated by reference herein.

The micelles of the instant invention are sometimes referred to herein as nanoparticles. For example, in the Example, the micelles are referred to as nanoparticles after cross-linking. Generally, the delivery vehicles of the instant invention will be micelles or have micellar characteristics (e.g., an aggregate of surfactant or amphiphilic molecules where, in an aqueous solution, the hydrophilic portion forms a shell and the hydrophobic portion forms the center or core of the micelle/particle) and also be nanoparticles due to their nanoscale size.

In certain embodiments, the delivery vehicle of the instant invention is up to about 2 or 3 μm in diameter (e.g., z-average diameter) or its longest dimension, particularly up to about 1 μm (e.g., about 1 nm to about 1 μm; nanoscale/nanoparticle). For example, the diameter or longest dimension of the delivery vehicle may be about 1 to about 800 nm. In certain embodiments, the diameter or longest dimension of the delivery vehicle is about 1 to about 750 nm, about 5 to about 500 nm, about 10 nm to about 300 nm, about 25 nm to about 250 nm, or about 50 nm to about 200 nm. In certain embodiments, the diameter or longest dimension of the delivery vehicle is less than about 1 μm, less than about 500 nm, less than about 300 nm, less than about 200 nm, or less than about 100 nm.

The polymers of the delivery vehicle (e.g., micelle) may be a block copolymer. In certain embodiments, the block copolymer comprises an ionically charged polymeric segment/block and a non-ionically charged polymeric segment/block (e.g., hydrophilic segment/block). In certain embodiments, the ionic polymeric segment is grafted with a hydrophobic moiety (or moieties). The hydrophobic-modified ionically charged polymeric segment forms the core of the micelle and the non-ionically charged polymeric segment forms the shell of the micelle.

The block copolymer has the structure A-B or B-A. The block copolymer may also comprise more than 2 blocks. For example, the block copolymer may have the structure A-B-A, wherein B is an ionically charged polymeric segment and A is a non-ionically charged polymeric segment. In a particular embodiment, the segments of the block copolymer comprise about 20 to about 300 repeating units, about 50 to about 250 repeating units, about 75 to about 200 repeating units, or about 100 to about 175 repeating units.

The ionically charged polymeric segment may be cationic or anionic. The ionically charged polymeric segment may be selected from, without limitation, polymethylacrylic acid and its salts, polyacrylic acid and its salts, copolymers of acrylic acid and its salts, poly(phosphate), polyamino acids (e.g., polyglutamic acid, polyaspartic acid), polymalic acid, polylactic acid, homopolymers or copolymers or salts thereof of aspartic acid, 1,4-phenylenediacrylic acid, ciraconic acid, citraconic anhydride, trans-cinnamic acid, 4-hydroxy-3-methoxy cinnamic acid, p-hydroxy cinnamic acid, trans glutaconic acid, glutamic acid, itaconic acid, linoleic acid, linlenic acid, methacrylic acid, maleic acid, trans-β-hydromuconic acid, trans-trans muconic acid, oleic acid, vinylsulfonic acid, vinyl phosphonic acid, vinyl benzoic acid, and vinyl glycolic acid and the like and carboxylated dextran, sulfonated dextran, heparin and the like. Examples of polycationic segments include—but are not limited to—polymers and copolymers and their salts comprising units deriving from one or several monomers including, without limitation: primary, secondary and tertiary amines, each of which can be partially or completely quaternized forming quaternary ammonium salts. Examples of these monomers include, without limitation, cationic amino acids (e.g., lysine, arginine, histidine), alkyleneimines (e.g., ethyleneimine, propyleneimine, butileneimine, pentyleneimine, hexyleneimine, and the like), spermine, vinyl monomers (e.g., vinylcaprolactam, vinylpyridine, and the like), acrylates and methacrylates (e.g., N,N-dimethylaminoethyl acrylate, N,N-dimethylaminoethyl methacrylate, N,N-diethylaminoethyl acrylate, N,N-diethylaminoethyl methacrylate, t-butylaminoethyl methacrylate, acryloxyethyltrimethyl ammonium halide, acryloxyethyl-dimethylbenzyl ammonium halide, methacrylamidopropyltrimethyl ammonium halide and the like), allyl monomers (e.g., dimethyl diallyl ammoniam chloride), aliphatic, heterocyclic or aromatic ionenes. Examples of non-ionically charged water soluble polymeric segments include, without limitation, polyetherglycols, poly(ethylene oxide), copolymers of ethylene oxide and propylene oxide, polysaccharides, polyvinyl alcohol, polyvinyl pyrrolidone, polyvinyltriazole, N-oxide of polyvinylpyridine, N-(2-hydroxypropyl)methacrylamide (HPMA), polyortho esters, polyglycerols, polyacrylamide, polyoxazolines, polyacroylmorpholine, and copolymers or derivatives thereof.

In certain embodiments, the ionically charged polymeric segment is polyglutamic acid. In certain embodiments, the non-ionically charged polymeric segment is poly(ethylene glycol) (PEG). In certain embodiments, the ionically charged polymeric segment is polyglutamic acid and the non-ionically charged polymeric segment is poly(ethylene glycol) (PEG).

The ionically charged segment of the polymers of the instant invention may comprise at least one hydrophobic moiety. For example, the ionically charged segment may be grafted with one or more hydrophobic moieties. The hydrophobization of the ionically charged segment yields an amphiphilic block copolymer with a non-ionically charged water soluble (hydrophilic) polymeric segment. In a particular embodiment, the degree of grafting of the hydrophobic moiety is at least about 20%, at least about 25%, at least about 30%, at least about 35%, at least about 40%, at least about 50%, at least about 60%, at least about 70%, at least about 80%, at least about 90%, or more. The hydrophobic moiety can be coupled to the ionically charged segment by any means including, for example, linking with functional groups of the ionically charged segment. The hydrophobic moiety may be linked directly to the ionically charged segment or via a linker. Generally, the linker is a chemical moiety comprising a covalent bond or a chain of atoms that covalently attaches the ligand to the surfactant. The linker can be linked to any synthetically feasible position of the hydrophobic moiety and the ionically charged segment. The linker may be degradable (e.g., substantially cleaved under physiological environments or conditions) or non-degradable. The linker may be a covalent bond or any other chemical structure which cannot be substantially cleaved or cleaved at all under physiological environments or conditions. In certain embodiments, the linker is PEG.

The hydrophobic moiety may be a compound with a relatively low molecular weight (e.g., less than 4,000, less than 2,000, or less than 1 kDa or 800 Da). In certain embodiments, the hydrophobic moiety is a lipid, fatty acid (saturated or unsaturated), steroid, or cholesterol. In certain embodiments, the hydrophobic moiety is a hydrophobic amino acid such as Val, Ile, Leu, Ala, Met, Phe, Trp, or Tyr—particularly phenylalanine. In certain embodiments, the hydrophobic moiety comprises at least one linear, branched or cyclic alkyl group, alkenyl group, and/or at least one aryl group.

The polymer micelles of the instant invention may self-assemble by neutralizing the ionically-charged polymeric segments with moieties of opposite charge. The neutralization of the charge allows for the creation of a hydrophobic core and hydrophilic shell formation of a micelle. In a particular embodiment, the neutralizing agent binds well and forms a complex with the polyionic segment, but is also easily removed (e.g., by dialysis, chromatography, ultrafiltration, centrifugation, or other means known in the art) and compatible with micelle chemistry. In a particular embodiment, the neutralizing agent is an ion or salt (e.g., metal ion) or a surfactant. The ion may be a mono-, di-, tri-, or multivalent ion. Examples of cations include, for example, Ca⁺², Mg⁺², Ba⁺² and Sr⁺² or multivalent cations such as spermine, spermidine, and the like. Examples of anions include, without limitation, Cl⁻ and Br⁻. Surfactants that may be used as neutralizing agents include, without limitation single-, double- or triple-tailed surfactants. Examples of cationic surfactants and anionic surfactants are provided in U.S. Pat. No. 7,332,527.

The cores of the micelles of the instant invention may be cross-linked. In a particular embodiment, the degree of cross-linking is at least about 5%, at least about 10%, at least about 15%, at least about 20%, at least about 25%, at least about 30%, at least about 50%, or more. The cross-linking of the inner core prevents the micelle from degradation upon dilution. Further, the biological agents contained within the core are protected from premature release and degradation. The hydrophilic outer shell of the micelles provides increased solubility and reduces unwanted interactions with blood plasma components.

The term “cross-linker” refers to a molecule capable of forming a covalent linkage between compounds (e.g., polymers) or between two different regions of the same compound (e.g., polymer). In certain embodiments, the cross-linker forms covalent linkages among the ionically charged polymeric segment, is compatible with micelle chemistry, and excess cross-linker is also easily removed (e.g., by dialysis or other means known in the art). Cross-linkers are well known in the art. In certain embodiments, the cross-linker is a titrimetric cross-linking reagent. The cross-linker may be a bifunctional, trifunctional, or multifunctional cross-linking reagent. Examples of cross-linkers are provided in U.S. Pat. No. 7,332,527. Cross-linking of the ionic core domain can be achieved by a variety of means including, without limitation, condensation reactions, addition reactions, or chain polymerization reactions (e.g., cationic chain polymerization, anionic chain polymerization, radical chain polymerization, and ring opening chain polymerization). Cross-linking may be achieved, without limitation, photochemically, spontaneously, by addition of a chain polymerization initiator, or by addition of titrimetric cross-linking reagents. Titrimetric cross-linkers can have a variety of functional groups useful in reacting with functionalities on the amphiphilic copolymers such as, without limitation, nucleophilic groups, electrophilic groups, and groups which participate in pericyclic reactions. Titrimetric cross-linkers include, without limitation, multifunctional compounds such as polyols, polyamines, polyethyleneglycol multiarm stars, polycarboxylic acids, polycarboxylic acid halides, polyisocyanates, polymeric aromatic isocyanates, polyalkylhalides, polysulfonates, polysulfates, polyphosphonates, polyphosphates, alkyldiamines, alkanediols, ethanolamine, poly(oxyethylene), amino-substituted poly(oxyethylene), diamino-substituted poly(oxyethylene), poly(ethyleneimine), polyamino-substituted poly(oxyethylene), amino-substituted alcohols, substituted dendrimers, and substituted hyperbranched polymers.

The cross-linked micelles of the instant invention are stable and control diffusion of the encapsulated compound(s). The rate of diffusion can be controlled the properties of cross-linked core of the micelle by, for example, the nature of cross-linking agent, the degree of cross-linking, and/or the composition of polyion-metal complex. Of course, the micelle must also release the entrapped compound(s) at the target site. In a particular embodiment, the cross-linker is reversible and/or biodegradable. In a particular embodiment, the cross-linker comprises a bond which may be cleaved in response to chemical stimuli (e.g., a disulfide bond that is degraded in the presence of intracellular glutathione). The cross-linkers may also be sensitive to pH (e.g., low pH).

In certain embodiments, the micelles are synthesized by at least partially hydrophobizing the ionically-charged polymeric segment of at least one block polymer having at least one ionically-charged polymeric segment and at least one non ionically-charged polymeric segment (hydrophilic); neutralizing the ionically-charged polymeric segments with moieties of opposite charge (e.g., a metal ion (e.g., Ca⁺²) or a surfactant) under conditions that allow for self-assembly of polymer micelles; cross-linking the neutralized ionically-charged polymer segments with a cross-linking agent; and removing the moieties of opposite charge and unreacted cross-linking agent.

The delivery vehicles of the instant invention (e.g., micelles) may be modified or conjugated to at least one targeting moiety, particularly on the outer portion of the delivery vehicle. A targeting moiety is a compound that will specifically bind to a specific type of tissue or cell type. The targeting moiety may be any type of molecule that binds to a specific cell or tissue type including but not limited to a small molecule, an antibody, an antibody fragment, a protein, or a peptide. In a particular embodiment, the targeting moiety is a ligand for a cell surface marker/receptor. The targeting moiety may be an antibody or fragment thereof immunologically specific for a cell surface marker (e.g., protein or carbohydrate) preferentially or exclusively expressed on the targeted tissue or cell type. The targeting moiety can be coupled to the micelles by any means including, for example, linking with functional groups of the non-ionic polymeric shell segments. The targeting moiety may be linked directly to the delivery vehicle or via a linker. Generally, the linker is a chemical moiety comprising a covalent bond or a chain of atoms that covalently attaches the moiety to the delivery vehicle. The linker can be linked to any synthetically feasible position of the targeting moiety and the delivery vehicle (e.g., the non-ionic polymeric shell segment of the micelle). Exemplary linkers may comprise at least one optionally substituted; saturated or unsaturated; linear, branched or cyclic alkyl group or an optionally substituted aryl group. The linker may also be a polypeptide (e.g., from about 1 to about 10 amino acids, particularly about 1 to about 5). The linker may be degradable or non-degradable. The linker may be a covalent bond or any other chemical structure which cannot be substantially cleaved or cleaved at all under physiological environments or conditions. In certain embodiments, the linker is PEG.

In certain embodiments, the targeting moiety targets and/or binds to leukocytes. In certain embodiments, the targeting moiety binds to monocytes and/or macrophages. In certain embodiments, the targeting moiety binds to a specific receptor found on macrophages and/or monocytes including, but not limited to: sialoadhesin receptors, folate receptors (e.g., folate (folic acid) and folate receptor antibodies and fragments thereof (see, e.g., Sudimack et al. (2000) Adv. Drug Del. Rev., 41:147-162)), galactose receptors, mannose receptors (e.g., mannose), formyl peptide receptor (FPR) ligands (e.g., N-formyl-Met-Leu-Phe (fMLF)) beta glucan receptors, scavenger receptors, hyaluronan receptors, Fc receptors, and tuftsin receptors (e.g., neuropilin-1 (Nrp1)). In certain embodiments, the targeting moiety is selected from tuftsin peptide (amino acid sequence Thr-Lys-Pro-Arg (SEQ ID NO: 1)) and/or analogs of the tuftsin peptide, tuftsin receptor antibodies and/or antibody fragments, folate, dextran, glycan, mannose, mannose derivatives and analogs, hyaluronic acid, GGP peptide (amino acid sequence Gly-Gly-Pro-Asn-Leu-Thr-Gly-Arg-Trp (SEQ ID NO: 2)), RGD peptide (amino acid sequence Arg-Gly-Asp (SEQ ID NO: 3); or a cyclic RGD (cRGD), internalizing RGD (iRGD), or RGD mimic/analog (see, e.g., European Patent Application EP2239329; U.S. Patent Application Publication NO. 2010/0280098)), CD11b antibody, CD14 antibody, F4/80 antibody, CX3CR1 antibody, Triggering Receptor Expressed on Myeloid Cells-1 (TREM1) antibody, TREM2 antibody, and macrophage colony stimulating-factor receptor (CD115) antibody. In certain embodiments, the targeting moiety is tuftsin.

The delivery vehicle (e.g., micelle) of the instant invention can encapsulate at least one compound. The compound(s) can be, without limitation, a biological agent, imaging/detection agent, and/or therapeutic agent. The encapsulated compounds include, without limitation, bioactive agents, therapeutics, diagnostics, nucleic acid molecules, DNA (e.g., oligonucleotides and plasmids), RNA (e.g., RNAi), proteins, polypeptides, polysaccharides, small molecules, and the like. The compounds may be stabilized within the core by non-covalent electrostatic and/or hydrophobic and/or nonpolar interactions. The ionic character of the core allows for the encapsulation of various charged molecules including, without limitation, both low molecular mass and biological agents such as small molecules, oligo- and polysaccharides, polypeptides and proteins, nucleic acid molecules (e.g., polynucleotides, siRNA, antisense molecules, etc.), and the like. Insoluble and hydrophobic agents can be immobilized through the interactions with hydrophobic groups in the core. The complexed micelles of the instant invention remain stable in aqueous dispersion due to the effect of hydrophilic exterior shell chains.

As used herein, the term “bioactive agent” also includes compounds to be screened as potential leads in the development of drugs or plant protecting agents. Bioactive agent and therapeutic agents include, without limitation, polypeptides, peptides, glycoproteins, nucleic acids, synthetic and natural drugs, peptoides, polyenes, macrocyles, macrolides, glycosides, terpenes, terpenoids, aliphatic and aromatic compounds, small molecules, and their derivatives and salts. The therapeutic agent may be a chemical compound such as a synthetic or natural drug. Imaging and detectable agents include, without limitation, contrast agents, paramagnetic or superparamagnetic ions for detection by MRI imaging, isotopes (e.g., radioisotopes (e.g., ³H (tritium) and ¹⁴C) or stable isotopes (e.g., ²H (deuterium), ¹¹C, ¹³C, ¹⁷O and ¹⁸O), optical agents, and fluorescence agents. Fluorescent agents include, without limitation, fluorescein and rhodamine and their derivatives. Optical agents include, without limitation, derivatives of phorphyrins, anthraquinones, anthrapyrazoles, perylenequinones, xanthenes, cyanines, acridines, phenoxazines and phenothiazines.

In certain embodiment, the delivery vehicles of the instant invention comprise or encapsulate an inhibitor of oxidative phosphorylation. In certain embodiment, the delivery vehicles of the instant invention comprise or encapsulate an inhibitor of ATP synthase. In certain embodiments, the delivery vehicles of the instant invention comprise or encapsulate an electron transport inhibitor. Examples of inhibitors of oxidative phosphorylation include but are not limited to an oligomycin (e.g., oligomycin A, oligomycin B, oligomycin C, oligomycin D (Rutamycin A), oligomycin E, or oligomycin F), rutamycin B, 44-homooliomycin A, 44-homooligomycin B, a leucinostatin (e.g., leucinostatin A, B, C, D, H, or K), an efrapeptin (e.g., efrapeptin C, D, E, F, or G), resveratrol, piceatannol, (−)epigallocatechin gallate, (−)epicatechin gallate, quercetin, and genistein. In certain embodiments, the encapsulated compound is an oligomycin, particularly oligomycin A.

The instant invention encompasses compositions comprising at least one delivery vehicle (e.g., micelle) of the instant invention and at least one pharmaceutically acceptable carrier. The compositions of the instant invention may further comprise other therapeutic agents (e.g., an antimicrobial or antibiotic). As used herein, the term “antibiotic” refers to antibacterial agents for use in mammalian, particularly human, therapy. Antimicrobial or antibacterial (antibiotics) include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof. In certain embodiments, the antimicrobial or antibacterial (antibiotics) is selected from the group consisting of vancomycin, rifampin, daptomycin, linezolid, tigecycline, quinupristin/dalfopristin (Synercid®), trimethoprim-sulfamethoxazole, clindamycin, tetracycline, doxyclycline, minocycline, delafloxacin, lefamulin, fosomycin, cefiderocol, plaxomicin, omadacycline, iclaprim, relebactam, eravacycline, meropenem, vaborbactam, dicloxacillin, flucloxacillin, amoxicillin/clavulanate, ticarcillin/clavulanate, piperacillin/tazobactam, cephazolin, cephalothin, cephalexin, teicoplanin, fusidic acid, ciprofloxacin, moxifloxacin, gatifloxacine, levofloxacin, gentamicin, cefepime, aztreonam, and imipenem.

The present invention also encompasses methods for preventing, inhibiting, and/or treating a bacterial infection. The methods comprise administering at least one delivery vehicle of the instant invention to a subject in need thereof. The method may further comprise administering at least one antimicrobial or antibiotic (e.g., systemic administration of the antimicrobial or antibiotic). The delivery vehicle (optionally as a pharmaceutical composition) of the instant invention can be administered to an animal (such as livestock and companion animals), in particular a mammal, more particularly a human, in order to treat/inhibit/prevent the bacterial infection. The pharmaceutical compositions of the instant invention may also comprise at least one antimicrobial or antibiotic. The antimicrobial or antibiotic may also be administered in a separate composition from the delivery vehicles of the instant invention. The compositions may be administered at the same time or at different times (e.g., sequentially). In certain embodiments, the delivery vehicle is administered prior to the antimicrobial or antibiotic. In certain embodiments, the delivery vehicle is administered one to fourteen days prior to administration of antimicrobial or antibiotic. In certain embodiments, the delivery vehicle is administered one to seven days prior to administration of antimicrobial or antibiotic.

In certain embodiments, the bacterial infection comprises a biofilm. Generally, the term “biofilm” refers to an established aggregation or community of bacteria on a biotic and/or abiotic surface with a matrix of extracellular polymeric substance secreted by the bacteria, thereby forming a film-like structure. For avoidance of any doubt, the term “biofilm” is not intended to include a mere bacteria cluster or bacteria in a planktonic slate. Those skilled in the art can readily detect the presence of an established biofilm using known techniques. In certain embodiments, the bacterial infection is at an implant within a subject. In certain embodiments, the bacterial infection is a prosthetic joint infection.

The biofilm infections may comprise gram positive bacteria or gram negative bacteria. In a particular embodiment, the bacterial infection comprises a gram positive bacteria biofilm (e.g., a Staphylococcus aureus biofilm). Gram-positive bacteria include, but are not limited to: Staphylococcus spp (e.g., Staphylococcus aureus (including MRSA)), Enterococcus spp (e.g., Enterococcusfecalis), Listeria monocytogenes, Bacillus spp, Lactobacillus plantarum, Lactococcus lactis, Streptococcal spp (e.g., Streptococcus pneumoniae and Streptococcus mutans). Gram-negative bacteria include, but are not limited to: Pseudomonas aeruginosa, Acinetobacter baumannii, Escherichia coli, Enterobacter spp, Klebsiella pneumoniae and Pantoea agglomerans. In certain embodiments, the bacteria is an antibiotic-resistant bacteria or an ESKAPE pathogen. In certain embodiments, the bacteria is selected from the group consisting of Enterococcus faecium, Staphylococcus aureus, Klebsiella pneumoniae, Acinetobacter baumannii, Pseudomonas aeruginosa, and Enterobacter species.

The dosage ranges for the administration of the compositions of the invention are those large enough to produce the desired effect. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The delivery vehicles described herein will generally be administered to a patient as a pharmaceutical preparation. The term “patient” as used herein refers to human or animal subjects. These delivery vehicles may be employed therapeutically, under the guidance of a physician.

The compositions comprising the delivery vehicles of the instant invention may be conveniently formulated for administration with any pharmaceutically acceptable carrier(s). For example, the delivery vehicles may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the delivery vehicles in the chosen medium may be varied and the medium may be chosen based on the desired route of administration of the pharmaceutical preparation. Except insofar as any conventional media or agent is incompatible with the delivery vehicles to be administered, its use in the pharmaceutical preparation is contemplated.

The dose and dosage regimen of delivery vehicles according to the invention that are suitable for administration to a particular patient may be determined by a physician considering the patient's age, sex, weight, general medical condition, and the specific condition for which the micelles are being administered and the severity thereof. The physician may also take into account the route of administration, the pharmaceutical carrier, and the biological activity of the delivery vehicles.

Selection of a suitable pharmaceutical preparation will also depend upon the mode of administration chosen. For example, the micelles of the invention may be administered by direct injection (e.g., to the site of infection and/or to the surrounding area) or intravenously. In this instance, a pharmaceutical preparation comprises the delivery vehicles dispersed in a medium that is compatible with the site of injection. The biofilm infections may occur at various sites within a patient including, but not limited to: catheters, joint prosthetics/implants, heart valves, sinus tissue, teeth, gums, urinary tract, lungs, and other tissue or implanted devices.

Delivery vehicles of the instant invention may be administered by any method. For example, the delivery vehicles of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In certain embodiments, the delivery vehicles are administered directly to the site of infection and/or the surrounding area. In certain embodiments, the delivery vehicles are administered at or near an infected implant/prosthetic device, within an infected catheter, and/or within an infected area of tissue. Pharmaceutical preparations for injection are known in the art. If injection is selected as a method for administering the delivery vehicles, steps must be taken to ensure that sufficient amounts of the molecules reach their target cells to exert a biological effect. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a delivery vehicle of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art. In certain embodiments, the delivery vehicle is administered with a concentration of therapeutic agent (e.g., inhibitor of oxidative phosphorylation) of about 0.1 μg/kg to about 100 μg/kg, about 0.5 μg/kg to about 25 μg/kg, about 1 μg/kg to about 10 μg/kg, about 3 μg/kg to about 5 μg/kg, or about 4 μg/kg.

In accordance with the present invention, the appropriate dosage unit for the administration of delivery vehicles may be determined by evaluating the toxicity of the molecules or cells in animal models. Various concentrations of delivery vehicles in pharmaceutical preparations may be administered to mice, and the minimal and maximal dosages may be determined based on the beneficial results and side effects observed as a result of the treatment. Appropriate dosage unit may also be determined by assessing the efficacy of the delivery vehicle treatment in combination with other standard drugs. The dosage units of delivery vehicle may be determined individually or in combination with each treatment according to the effect detected.

The pharmaceutical preparation comprising the delivery vehicles may be administered at appropriate intervals, for example, at least twice a day or more until the pathological symptoms are reduced or alleviated, after which the dosage may be reduced to a maintenance level. The appropriate interval in a particular case would normally depend on the condition of the patient. In certain embodiments, the delivery vehicles of the instant invention are administered only once to the subject.

### Definitions

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., bacterial infection) resulting in a decrease in the probability that the subject will develop the condition.

As used herein, the term “subject” refers to an animal, particularly a mammal, particularly a human.

A “therapeutically effective amount” of a compound or a pharmaceutical composition refers to an amount effective to prevent, inhibit, treat, and/or lessen the symptoms of a particular disorder or disease. The treatment of a microbial infection (e.g., a bacterial infection such as a S. aureus infection) herein may refer to an amount sufficient to inhibit microbial growth or kill the microbe and/or curing, relieving, and/or preventing the microbial infection, the symptom of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

The term “antimicrobials” as used herein indicates a substance that kills or inhibits the growth of microorganisms such as bacteria, fungi, viruses, or protozoans, particularly bacteria.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

As used herein, a “linker” is a chemical moiety comprising a covalent bond or a chain of atoms that covalently attaches two molecules to each other. In certain embodiments, the linker comprises amino acids, particularly from 1 to about 25, 1 to about 20, 1 to about 15, 1 to about 10 amino acids, or 1 to about 5 amino acids. In certain embodiments, the linker comprises at least one optionally substituted; saturated or unsaturated; linear, branched or cyclic alkyl or aliphatic group or an optionally substituted aryl group. In certain embodiment, the linker may contain from 0 (i.e., a bond) to about 50 atoms, from 0 to about 10 atoms, or from about 1 to about 5 atoms. The linker may be a lower alkyl or aliphatic. The term “lower alkyl” or “lower aliphatic” refers to an alkyl or aliphatic, respectively, which contains 1 to 3 carbons in the hydrocarbon chain. In a particular embodiment, the linker is PEG.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and antigen-binding fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The term “alkyl,” as employed herein, includes straight, branched, and cyclic chain hydrocarbons containing 1 to about 20 carbons or 1 to about 10 carbons in the normal chain. The hydrocarbon chain of the alkyl groups may be interrupted with one or more oxygen, nitrogen, or sulfur. Examples of suitable alkyl groups include methyl, ethyl, propyl, isopropyl, butyl, t-butyl, isobutyl, pentyl, hexyl, isohexyl, heptyl, 4,4 dimethylpentyl, octyl, 2,2,4 trimethylpentyl, nonyl, decyl, the various branched chain isomers thereof, and the like. Each alkyl group may, optionally, be substituted, preferably with 1 to 4 substituents. The term “lower alkyl” refers to an alkyl which contains 1 to 3 carbons in the hydrocarbon chain. The term “cyclic alkyl” or “cycloalkyl,” as employed herein, includes cyclic hydrocarbon groups containing 1 to 3 rings which may be fused or unfused. Cycloalkyl groups may contain a total of 3 to 20 carbons forming the ring(s), particularly 6 to 10 carbons forming the ring(s). Optionally, one of the rings may be an aromatic ring as described below for aryl. The cycloalkyl groups may also, optionally, contain substituted rings that includes at least one (e.g., from 1 to about 4) sulfur, oxygen, or nitrogen heteroatom ring members. Each cycloalkyl group may be, optionally, substituted, with 1 to about 4 substituents. Alkyl substituents include, without limitation, alkyl, alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. In a particular embodiment, the substituent is hydrophobic such as an alkyl or aryl.

“Alkenyl” refers to an unsubstituted or substituted hydrocarbon moiety comprising one or more carbon to carbon double bonds (i.e., the alkenyl group is unsaturated) and containing from 1 to about 20 carbon atoms or from 1 to about 10 carbon atoms, which may be a straight, branched, or cyclic hydrocarbon group. The hydrocarbon chain of the alkenyl groups may be interrupted with one or more oxygen, nitrogen, or sulfur. When substituted, alkenyl groups may be substituted at any available point of attachment. Exemplary substituents are described above for alkyl groups.

The term “aryl,” as employed herein, refers to monocyclic and bicyclic aromatic groups containing 6 to 10 carbons in the ring portion. Examples of aryl groups include, without limitation, phenyl, naphthyl, such as 1-naphthyl and 2-naphthyl, indolyl, and pyridyl, such as 3-pyridyl and 4-pyridyl. Aryl groups may be optionally substituted through available carbon atoms, preferably with 1 to about 4 groups. Exemplary substituents are described above for alkyl groups. The aryl groups may be interrupted with one or more oxygen, nitrogen, or sulfur heteroatom ring members (e.g., a heteroaryl).

The following example provides illustrative methods of practicing the instant invention and is not intended to limit the scope of the invention in any way.

### Example

### Methods

### Mice

Male and female C57BL/6NCrl mice (RRID:IMSR_CRL:27; 8 weeks of age) were purchased from Charles River Laboratories (Frederick, Md.). When animals were designated for experiments, mice of the same sex were randomized into standard density cages with a total of 5 animals per cage. Mice were housed in a restricted-access BSL2 room equipped with ventilated microisolator cages and maintained at 21° C. under a 12 hours light:12 hours dark cycle with ad libitum access to water (Hydropac™; Lab Products, Seaford, Del.) and Teklad rodent chow (Harlan, Indianapolis, Ind.) with Nestlets provided for enrichment.

Mouse Model of S. aureus Orthopedic Implant Biofilm Infection

To model infectious complications that can arise in patients following arthroplasty, a mouse model of S. aureus orthopedic implant infection was used (Heim, et al. (2014) Methods Mol. Biol., 1106:183-191; Heim, et al. (2015) J. Leukoc. Biol., 98:1003-1013; Heim, et al. (2014) J. Immunol., 192:3778-3792; Heim, et al. (2015) J. Immunol., 194:3861-3872; Scherr, et al. (2014) Methods Mol. Biol., 1106:173-181). This model reflects biofilm growth as demonstrated by us and others using SEM and H&E staining (Heim, et al. (2015) J. Immunol., 194(8):3861-72; Heim, et al. (2014) J. Immunol., 192(8):3778-92; Niska, et al. (2012) PloS One, 7(10):e47397; Pribaz, et al. (2012) J. Orthop. Res., 30(3):335-40; Bernthal, et al. (2011) J. Orthop. Res., 29(10):1621-6; Thompson, et al. (2018) JCI Insight, 3(17): e121737). In addition, immunophenotyping of patients with PJI, many of which were diagnosed with S. aureus, has convincingly demonstrated similar leukocyte infiltrates as observed in the mouse model (Heim, et al. (2015) J. Immunol., 194(8):3861-72; Heim, et al. (2018) J. Orthop. Res., 36(6):1605-13), supporting its translational utility. Briefly, mice were anesthetized with ketamine/xylazine (100 mg/kg and 5 mg/kg, respectively) and the surgical site was disinfected with povidone-iodine. After a surgical plane of anesthesia was achieved, a medial incision was created through the quadriceps with lateral displacement to access the distal femur. A burr hole was made in the femoral intercondylar notch through the intramedullary canal using a 26-gauge needle, whereupon a pre-cut 0.8-cm orthopedic-grade Kirschner wire (0.6 mm diameter, Nitinol [nickel-titanium]; Custom Wire Technologies, Port Washington, Wis.) was inserted into the intramedullary canal, leaving ˜1 mm protruding into the joint space. The exposed wire surface was inoculated with 10³ CFU of S. aureus USA300 LAC13c (11) in 2 μL of 1×PBS. Following closure of the surgical site with absorbable nylon sutures, animals received s.c. Buprenex® (0.1 mg/kg; Reckitt Benckiser, Hull, U.K.) for the first 24 hours after surgery for pain relief. The health status of mice was regularly monitored throughout the course of infection, and all mice exhibited normal ambulation and no discernable pain behaviors. At the indicated time points post-infection, mice were euthanized using an overdose of inhaled isoflurane with cervical dislocation as a secondary physical method of euthanasia.

### Generation of Bone Marrow-Derived Macrophages

Macrophages were expanded from the bone marrow of both male and female C57BL/6NCrl mice as described (Yamada, et al. (2018) Infect. Immun., 86(7) e00206-18). At day in vitro 6, macrophages were harvested and seeded at 5×10⁴ cells/well in a 96-well plate. Following an overnight adherence period, macrophages were pre-treated with 10 ng/mL recombinant mouse (rm)IL-4 (Cat #574306 BioLegend, San Diego, Calif.) for 1 hour followed by exposure to various concentrations of oligomycin (Cat #11342 Cayman Chemical) for 24 hours, whereupon medium was collected to quantify TNF-α by cytometric bead array (Cat #552364, BD Biosciences) and arginase activity (Cat #MAK112, Millipore Sigma).

### Nanoparticle Synthesis and Characterization

Polymeric nanoparticles based on an amphiphilic block copolymer of poly(ethylene glycol)-b-poly(L-glutamic acid) (PEG-b-PGA) with pendant phenylalanine functionalities were synthetized as described (Desale, et al. (2015) J. Controlled Rel., 220(Pt B):651-9; Kim, et al. (2013) J. Drug Targeting, 21(10):981-93). Briefly, PEG-b-PGA (Alamanda Polymers, Inc., Madison, Ala.; block lengths of 114 and 150 repeating units for PEG and PGA, respectively) was first modified with L-phenylalanine methyl ester via carbodiimide chemistry. The degree of grafting was 50% as determined by ¹H-NMR analysis. Polymeric micelles were then prepared by mixing the copolymer solution in dimethylformamide with water (1:1 v/v) followed by dialysis against water for 48 hours. The formed micelles were cross-linked using 1,2-ethylenediamine in the presence of 1-(3-dimethylaminopropyl)-3-ethylcarbodiimide hydrochloride (EDC) with a targeted cross-link density of 20% (based on the molar ratio of cross-linker to carboxylic groups of the GA residues).

Tuftsin peptide with a cysteine residue at the C-terminus (TKPRC (SEQ ID NO: 4)) was synthesized on an automated solid-phase Liberty microwave peptide synthesizer (CEM, Matthews, N.C.) employing standard Fmoc chemistry using a Rink Amide resin (Nova Biochem). Sample purification (>95%) was performed on a Phenomenex (Torrance, Calif.) Jupiter 10 μm Proteo 250×4.6 mm C12 column using a water (0.1% formic acid)-acetonitrile (0.1% formic acid) gradient. HPLC/MS analyses were performed on a Waters (Milford, Mass.) e2695 system equipped with a Waters 2489 absorption detector and a Waters Q-Tof Micro electrospray ionization mass spectrometer.

Tuftsin targeting moieties were conjugated to nanoparticles via a heterobifunctional maleimide-PEG-amine linker (MAL-PEG-NH₂, 7.5 kDa, JenKem Technology, Plane TX). First, MAL-PEG-NH₂ (0.27 μmol, 0.15 eq with respect to the amount of carboxylic groups) was conjugated to the free carboxyl groups (1.75 μmol) of EDC-activated nanoparticles. Resulting constructs were purified using repeated ultrafiltration (MWCO 30,000, Millipore) at 2200 rpm for 15 minutes (3 washes). Purified peptide (0.31 μmol) was subsequently reacted with MAL-PEG functionalized nanoparticles in PBS at pH 7 for 2 hours. Unreacted MAL groups were quenched by β-mercaptoethanol and targeted nanoparticles were purified by dialysis against distilled water using a dialysis membrane (MWCO 3500 Da). The amount of peptide conjugated on the surface of the nanoparticles was 126.3±6.3 μg per mg of polymer (n=3) as determined by a BCA protein assay. Oligomycin-loaded nanoparticles were prepared by adding an ethanol solution of oligomycin (2 mg/mL; Cat #11342 Cayman Chemical, Ann Arbor, Mich.) dropwise into the aqueous dispersion of tuftsin-coated nanoparticles (1 mg/mL) and mixed overnight at room temperature in an open-air system to allow for the slow evaporation of ethanol, whereupon residual ethanol was removed at reduced pressure. Unincorporated oligomycin was removed by filtration with 0.8 μm syringe filters (Thermo Scientific). Oligomycin content was determined by HPLC analysis under isocratic conditions using an Agilent 1200 HPLC system with a diode array detector set at 226 nm (Zu, et al. (2011) Intl. J. Nanomed., 6:3429-41). A Nucleosil C18 column was used as stationary phase (250 mm×4.6 mm), and the mobile phase was comprised of an acetonitrile/water mixture (80/20, v/v) applied at a flow rate of 1 mL/minute. The resulting nanoparticles had an average hydrodynamic diameter of approximately 80 nm (ζ-potential=−8.4 mV) and were characterized by narrow size distributions using dynamic light scattering (Zetasizer Nano ZS, Malvern Instruments Ltd., Worcestershire, UK). The loading capacity of tuftsin-coated nanoparticles for oligomycin was 5.2±0.9% (w/w).

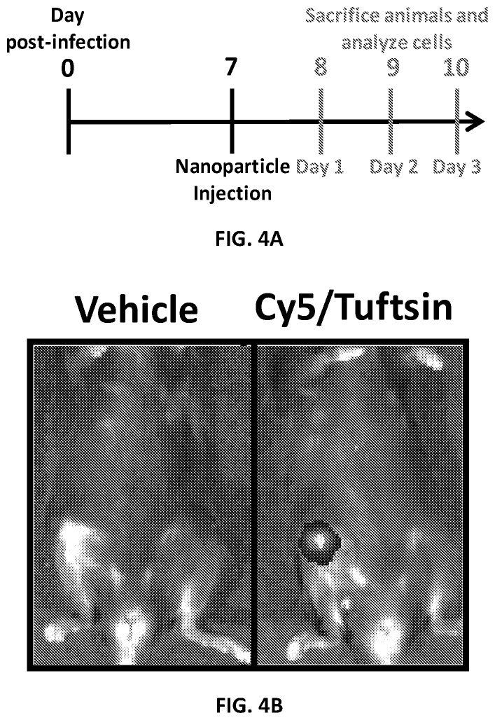

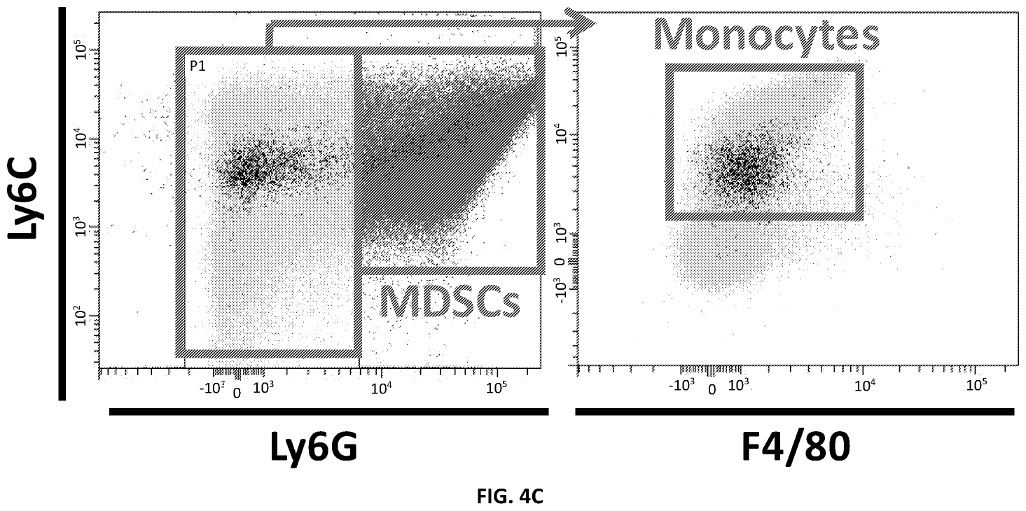

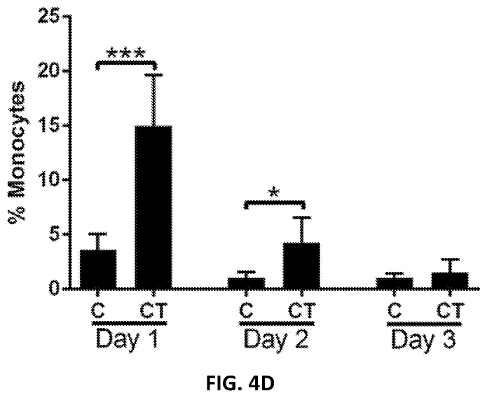

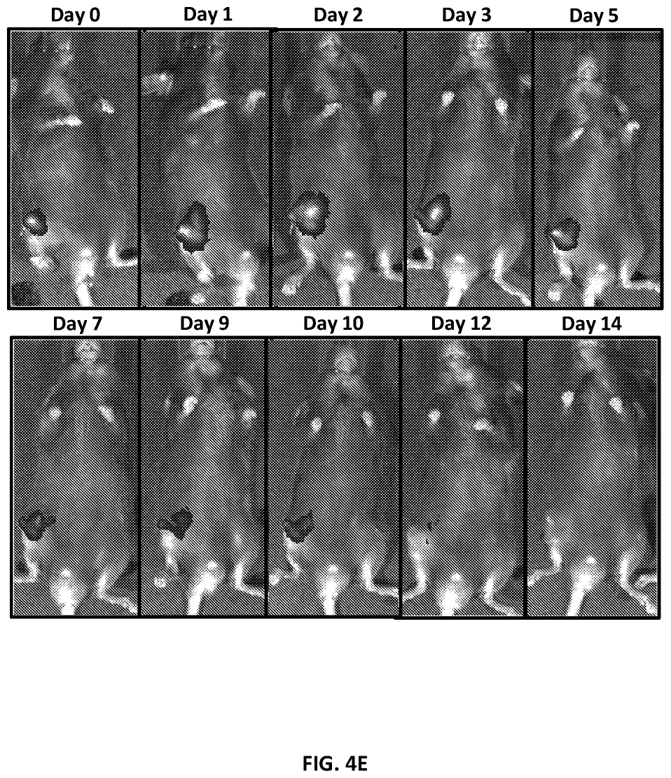

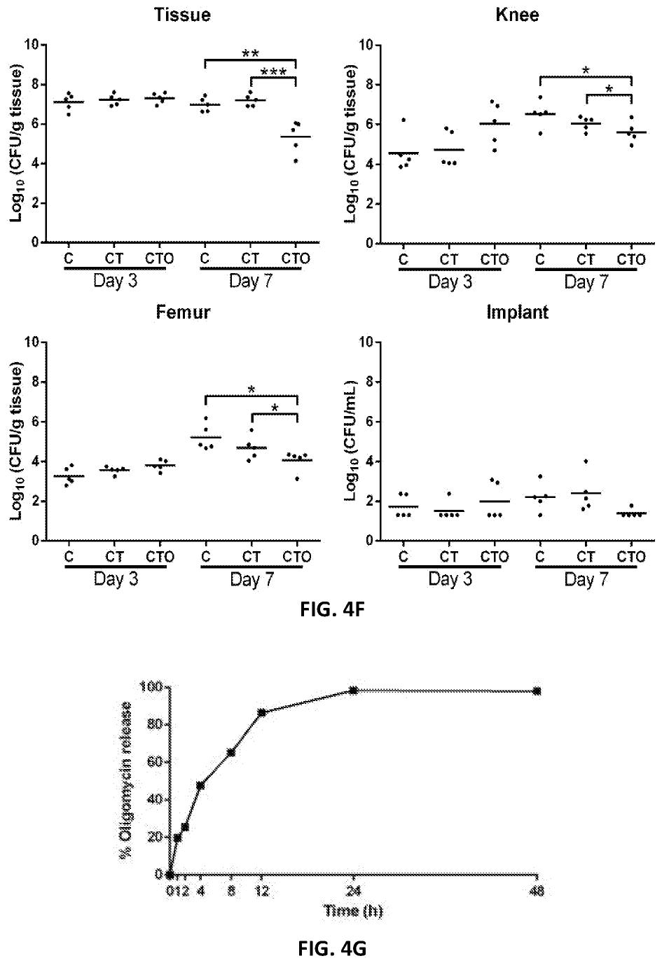

Finally, fluorescently labelled non-modified (C), tuftsin-coated empty (CT) and tuftsin-oligomycin-loaded (CTO) nanoparticles were synthesized by adding Cy5 amine (Lumiprobe Corporation, Hunt Valley, Md.) in DMSO to the aqueous dispersion of nanoparticles (equivalent to 0.16% of carboxylic groups in nanoparticles) in the presence of EDC and the mixture was incubated for 4 hours in the dark. Unbound dye was removed by dialysis and fluorescence emission spectra of labelled nanoparticles were characterized using a FluoroMax-4 spectrofluorometer (HORIBA Scientific). Oligomycin release from CTO nanoparticles was determined using a PBS dialysis method with a 3.5 kDa membrane cutoff. The kinetics of oligomycin release was determined by HPLC and concentrations are expressed as a percentage of the total oligomycin available vs. time. Over 90% of oligomycin was released from CTO nanoparticles within a 24 hour period at physiological pH (FIG. 4G).

### Nanoparticle Injection and IVIS Imaging

Animals were placed in an induction chamber without restraint and anesthetized with 2.5% isoflurane. A total of 10 μg control (C or CT) or oligomycin (CTO) nanoparticles were administered by a single intra-articular injection (in 10 μL of PBS) at either day 3 or 7 post-infection. Nanoparticle injection and length of retention was evaluated in the same cohort of mice using an In Vivo Imaging System (IVIS® Spectrum; PerkinElmer, Waltham, Mass.) under isoflurane anesthesia, with excitation and emission wavelengths of 640 nm and 680 nm, respectively.

Recovery of Infected Tissues for S. aureus Enumeration

The titanium implant, femur, knee joint, and surrounding soft tissue were collected by first removing the skin, whereupon the tissue ventral to the patellar tendon was excised, weighed, and disrupted using the blunt end of a 3 mL syringe in 500 μL of PBS supplemented with a protease inhibitor cocktail tablet (ThermoFisher). The remaining muscle and tendons were removed and excluded from analysis. The knee joint and femur were separated and homogenized individually using a hand-held homogenizer for 30 seconds. The titanium implant was carefully removed and vortexed in 200 μL PBS to dislodge biofilm-associated bacteria. S. aureus titers were quantified using TSA plates supplemented with 5% sheep blood (Cat #R01202, Remel, Lenexa, Kans.) and are expressed as Log₁₀ (cfu/mL) for titanium implant or Log₁₀ (cfu/g tissue) for tissues.

### JC-1 and 2-NBDG Staining

Leukocytes recovered from animals with sterile or S. aureus infected orthopedic implants were stained with the bi-potential dye JC-1 (5,5′,6,6′-Tetrachloro-1,1′,3,3′-tetraethylbenzimidazolocarbocyanine iodide; Cat #T3168 Invitrogen) or the fluorescent glucose analog 2-NBDG (2-(N-(7-Nitrobenz-2-oxa-1,3-diazol-4-yl)Amino)-2-Deoxyglucose; Cat #N13195 ThermoFisher) to evaluate OxPhos and glycolytic activity, respectively. JC-1 emits red fluorescence in mitochondria with large membrane potentials and green fluorescence in depolarized mitochondria. Therefore, cells exhibiting increased OxPhos activity have a larger green:red ratio. Cells were incubated with either 100 ng/mL JC-1 or 10 μM 2-NBDG for 30 minutes at 37° C. Pooled aliquots of cells from each mouse (n=5)±FCCP treatment were used as compensation controls. Cells were washed and resuspended in 250 μL FACS buffer supplemented with 2 μL Fc-block (TruStain FcX, Cat #101320 BioLegend, San Diego, Calif.) to minimize nonspecific antibody binding. Finally, cells were stained with Live/Dead Fixable Blue Dead Cell Stain (Cat #L23105 Invitrogen, Eugene, Oreg.), CD45-APC (Cat #103112), Ly6G-PacBlue (Cat #127612), F4/80-PE-Cy7 (Cat #123114), and CD11b-AF700 (Cat #101222) (all from BioLegend), or Ly6C-PerCP-Cy5.5 (Cat #560525, BD Pharmingen), to identify monocytes (CD11b^{high}Ly6G⁻ Ly6C⁺F4/80⁻) as described. OxPhos activity (JC-1) was calculated as the ratio of green:red monocytes and glycolysis was calculated as the percentage of 2-NBDG⁺ monocytes.

### Flow Cytometry

To characterize leukocyte infiltrates associated with soft tissues surrounding the infected knee, homogenates were first passed through a 35 μm filter (BD Falcon, BD Biosciences). The cellular filtrate was washed with PBS supplemented with 2% heat-inactivated FBS and collected by centrifugation (300×g, 5 minutes), whereupon RBCs were lysed using RBC Lysis Buffer (BioLegend). Cells were washed and resuspended in PBS containing 2% FBS and incubated with TruStain fcX™ (BioLegend) to minimize non-specific antibody binding. Cells were then stained with Live/Dead Fixable Blue Dead Cell Stain (Invitrogen, Eugene, Oreg.), CD45-PacBlue (BioLegend), Ly6G-PE (BioLegend), Ly6C-PerCP-Cy5.5 (Pharmingen), F4/80-PE-Cy7 (BioLegend), and CD11b-FITC (BioLegend). An aliquot of pooled cells was stained with isotype-matched control antibodies to assess the degree of non-specific staining per treatment group. For individual samples, 10,000-100,000 events were analyzed using BD FACSDiva™ software with cell populations expressed as the percentage of total viable CD45⁺ leukocytes.

### Recovery of MDSCs and Monocytes for RT-qPCR Analysis

Monocytes and MDSCs from the soft tissue surrounding the infected knee were sorted by FACS on a FACSAria™ (BD Biosciences) using the nantibody panel described above, whereupon RNA was immediately isolated using a RNeasy® Plus Micro Kit (Qiagen, Hilden, Germany). cDNA was synthesized using the iScript™ cDNA Synthesis Kit (Bio-Rad, Hercules, Calif.) and RT-qPCR was performed using TaqMan® primer/probe mixes (ThermoScientific) for the following genes: Arg-1, IL-10, triggering receptor expressed on myeloid cells 2 (Trem2), hypoxia-inducible factor-alpha (HIF1-α), inducible nitric oxide synthase (iNOS), TNF-α, triggering receptor expressed on myeloid cells 1 (Trem1) and GAPDH. Gene expression levels of monocytes and MDSCs from CTO treated animals were normalized to GAPDH and are presented as the fold-induction (2^{-ΔΔCt}) value relative to monocytes and MDSCs isolated from CT treated animals.

### Seahorse Biosciences Assay

The bio-energetic profiles of bone marrow-derived macrophages were assessed using a XF96 extracellular flux analyzer (Seahorse Biosciences, North Billerica, Mass.). Briefly, macrophages were seeded in XF96 plates at 5×10⁴ cells/well 24 hour prior to assay, whereupon cells were treated with 10 ng/mL rmIL-4, peptidoglycan (PGN)+IFN-γ (10 g/mL and 10 ng/mL, respectively), or CTO nanoparticles (1 μg/mL) for 24 hours at 37° C. One hour prior to assay, macrophages were incubated in bicarbonate-free DMEM (Sigma-Aldrich) supplemented with 2 mM L-glutamine and 1 mM pyruvate at 37° C. without CO₂. Glucose was included at 10 mM for mitochondrial stress tests. For mitochondrial stress tests, oxygen consumption rate (OCR) was measured following sequential exposure to oligomycin (1 μM), carbonilcyanide p-triflouromethoxyphenylhydrazone (FCCP, 1 μM), and rotenone (0.5 μM) (all from Sigma-Aldrich). At the beginning of the assay, the Seahorse instrument measured changes in oxygen concentration in immediate proximity to macrophages to determine the baseline oxygen consumption rate. Next, oligomycin was injected to inhibit adenosine triphosphate (ATP) production by blocking ATP synthase. Following oligomycin treatment, FCCP was injected, which is a membrane uncoupler that causes an increase in oxygen consumption without ATP generation. Finally, the complex I inhibitor rotenone, was injected to completely block mitochondrial respiration. By sequentially inhibiting different stages of the electron transport chain, well-established algorithms were used to calculate basal respiration, ATP production, maximal respiration, and non-mitochondrial oxygen consumption (Rogers, et al. (2011) PLoS One 6:e21746; Tan, et al. (2015) Anim. Nutr., 1:239-243). To measure glycolytic activity, macrophages were pre-incubated for 1 hour in medium lacking sodium bicarbonate, serum, and glucose prior to initiating the assay to reduce intracellular glucose stores. Next, cells were sequentially exposed to glucose (10 mM) and oligomycin (1 μM; both from Sigma-Aldrich), whereupon extracellular acidification rate (ECAR) measured. Total protein was quantified in each well following Seahorse assays to confirm lack of macrophage toxicity.

### Metabolomic Analysis

Monocytes (CD11b^{high}Ly6G⁻Ly6C⁺F4/80⁻) and MDSCs (CD11b^{high}Ly6G⁺Ly6C⁺F4/80⁻) were recovered from animals receiving CT or CTO nanoparticles by FACS as described (Shukla, et al. (2017) Cancer Cell, 32(3):392). Sorted cells were collected in a microcentrifuge tube and polar metabolites were extracted (Shukla, et al. (2017) Cancer Cell 32:392). Cell sorting by positive selection was not expected to elicit dramatic metabolic changes based on the rapid processing period and the fact that cells were maintained on ice to reduce their metabolic rate. In addition, the positive selection protocol was identical for monocytes and MDSCs recovered from CT and CTO animals, which would control for any potential effects of positive selection when making comparisons. Briefly, immediately after collection, monocytes and MDSCs were lysed using an 80% methanol/water mixture pre-chilled to −80° C. Cellular debris was removed by centrifugation at 13,000 rpm for 5 minutes at 4° C., whereupon supernatants containing metabolite extracts were collected and dried at room temperature using a speed vacuum concentrator (Eppendorf, Hamburg, Germany). Samples were resuspended in a 50% methanol/water mixture and analyzed using LC-MS/MS. A single reaction monitoring (SRM) LC-MS/MS method was used with positive/negative ion polarity switching on a Xevo® TQ-S mass spectrometer (Gunda, et al. (2016) PLoS One 11:e0154416). The metabolite peak areas were calculated using MassLynx™ 4.1 (Waters Inc.) and normalized to the respective cell counts of each sample. The resultant peak areas were subjected to relative quantification analyses by utilizing MetaboAnalyst 3.0 (www.metaboanalyst.ca). Fold-change values of metabolites were expressed as CTO relative to CT treated mice. Individual values exhibited a normal distribution and outliers were identified by Grubb's test, followed by a Student's t-test to determine significance. Principal component analysis, heatmap, and pathway impact analysis were also performed using MetaboAnalyst 3.0 software.

### Statistics

Significant differences between experimental groups were determined by a one-way ANOVA with Bonferroni's multiple comparisons. The statistical details for all experiments can be found in the accompanying figure legends along with the “n” and precision measures. Sample sizes were determined based on prior publications using groups that were sufficient to achieve a desired power of 0.85 and an alpha value of 0.05. All analyses were performed using GraphPad Prism version 6.04 (San Diego, Calif.; RRID:SCR_002798) and a p-value of <0.05 was considered statistically significant.

### Results

S. aureus Biofilm Infection Promotes a Shift Towards OxPhos Metabolism in Monocytes

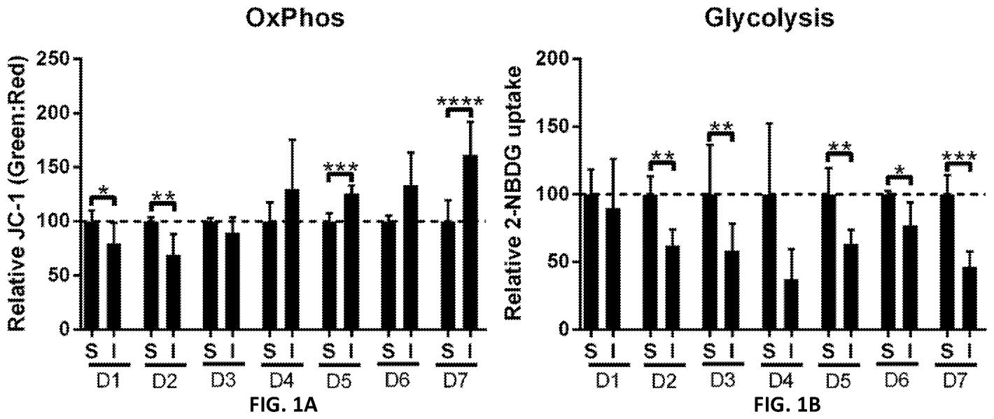

Using a mouse model of S. aureus PJI, it has been shown that biofilms promote monocyte anti-inflammatory activity (Heim, et al. (2015) J. Leukoc. Biol., 98(6):1003-13; Heim, et al. (2015) J. Immunol., 194(8):3861-72; Heim, et al. (2014) J. Immunol., 192(8):3778-92). To determine whether this anti-inflammatory bias is associated with a metabolic shift favoring OxPhos over glycolysis, the metabolic profiles of monocytes associated with S. aureus infected vs. sterile orthopedic implants were examined. The rationale for this comparison was two-fold; first, the placement of a sterile implant elicits a pro-inflammatory tissue injury response (Heim, et al. (2015) J. Immunol., 194(8):3861-72), which could be compared with the anti-inflammatory biofilm milieu. Second, evaluating the metabolic state of monocytes isolated from biofilms versus abscesses would lead to confounds because of differences in bacterial burdens between the models and the fact that abscesses are cleared, whereas biofilm infections remain chronic. Seahorse metabolic assays are often used to evaluate cellular metabolic states; however, this approach was not feasible given the limited numbers of implant-associated monocytes in vivo. Therefore, flow cytometry was performed with JC-1 and 2-NDBG, validated tools that are widely used in the immunometabolism field as indicators of cellular OxPhos and glycolytic activity, respectively (Perelman, et al. (2012) Cell Death Dis., 3:e430; Zou, et al. (2005) J. Biochem. Biophys. Methods, 64(3):207-15). JC-1 is a bi-potential dye that accumulates in mitochondria with large membrane potentials and fluoresces red, whereas depolarized mitochondria are green. Therefore, cells exhibiting increased OxPhos activity display a larger green:red ratio (Asmis, et al. (2003) Circ. Res., 92(1):e20-9). OxPhos was significantly increased in monocytes infiltrating S. aureus biofilms compared to sterile implants at later time points (i.e., days 5-7; FIG. 1A), which coincides with biofilm maturation as measured by antibiotic tolerance (Heim, et al. (2018) Infect. Immun., 86(12): e00684-18). Interestingly, monocyte OxPhos activity was significantly reduced during the first two days of infection (FIG. 1A), which may reflect an initial pro-inflammatory response that transforms over time to an anti-inflammatory state. Glycolysis was evaluated by the uptake of the fluorescent glucose analog 2-NBDG (Yamada, et al. (2007) Nat. Protoc., 2(3):753-62), which revealed significant and persistent reductions in glycolytic activity in S. aureus biofilm-associated monocytes compared to sterile implants (FIG. 1B). Collectively, increased OxPhos concomitant with reduced glycolysis in biofilm-associated monocytes corresponds with their anti-inflammatory properties.

### Inhibition of OxPhos by Oligomycin Promotes Macrophage Pro-Inflammatory Activity

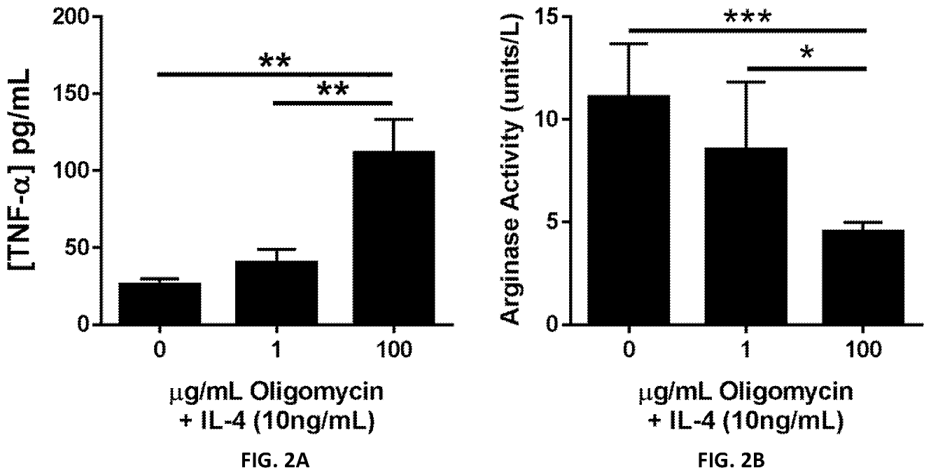

Since biofilm-associated monocytes displayed an OxPhos bias, it was explored whether this could be attenuated to promote their pro-inflammatory activity. Oligomycin inactivates mitochondrial ATP synthase of the electron transport chain to inhibit OxPhos (Izquierdo, et al. (2015) J. Immunol., 195(5):2442-51; Hao, et al. (2010) J. Biol. Chem., 285(17):12647-54). Prior work has demonstrated that oligomycin shifts macrophages towards aerobic glycolysis, which coincides with pro-inflammatory gene expression (Izquierdo, et al. (2015) J. Immunol., 195(5):2442-51). To establish the efficacy of oligomycin in reprogramming macrophage inflammatory properties, mouse bone marrow-derived macrophages were treated with the anti-inflammatory cytokine IL-4 in the presence/absence of oligomycin. Oligomycin reversed the anti-inflammatory effects of IL-4 by increasing TNF-α expression (FIG. 2A) and decreasing arginase activity (FIG. 2B) in a dose-dependent manner, hallmark molecules of pro- and anti-inflammatory macrophages, respectively (Wynn, et al. (2013) Nature 496(7446):445-55). Oligomycin was not toxic to macrophages, since protein yields were equivalent between cells with or without drug treatment.

### Oligomycin Nanoparticles Shift Macrophage Metabolism Towards Glycolysis

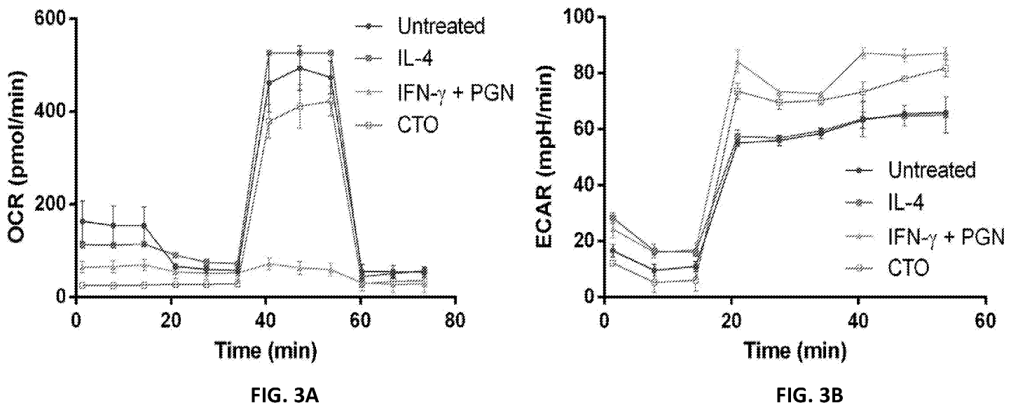

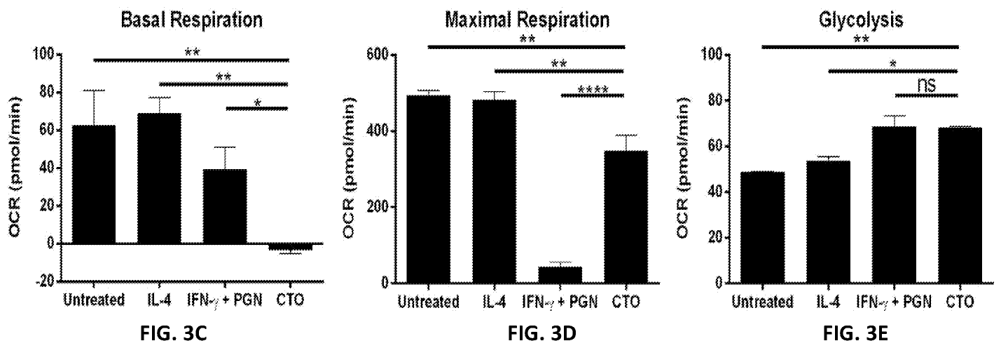

Oligomycin uptake was targeted to biofilm-associated monocytes in vivo to determine whether metabolic re-programming to aerobic glycolysis would enhance their pro-inflammatory activity and promote biofilm clearance. To achieve this goal, a nanoparticle delivery approach was designed using tuftsin as the targeting moiety. Tuftsin is a peptide derived from the Fc portion of IgG, which has been shown to facilitate nanoparticle internalization by macrophages through its interaction with Fc-receptors (Jain, et al. (2012) Biomacromolecules 13:1074-1085; Dutta, et al. (2008) Eur. J. Pharm. Sci., 34:181-189). Tuftsin-conjugated nanoparticles have been used to target monocytes and macrophages to deliver a variety of biomolecules in the context of arthritis, HIV, and other inflammatory diseases (Jain, et al. (2012) Biomacromolecules 13:1074-1085; Jain, et al. (2015) Biomaterials 61:162-177; Dutta, et al. (2008) Eur. J. Pharm. Sci., 34:181-189; Talekar, et al. (2015) AAPS J., 17:813-827). Three nanoparticle types were used, namely Cy5 (C), Cy5/Tuftsin (CT), and Cy5/Tuftsin/Oligomycin (CTO), where the former two represented control formulations and all three harbored the Cy5 fluorochrome to allow nanoparticle tracking (Table 1). Although oligomycin affects macrophage respiratory capacity through inhibition of mitochondrial ATP synthase (Izquierdo, et al. (2015) J. Immunol., 195:2442-2451), no studies have examined the efficacy of oligomycin in a nanoparticle formulation. To establish the effects of oligomycin nanoparticles on macrophage activity, bone marrow-derived macrophages were treated with prototypical anti-inflammatory (IL-4) and pro-inflammatory (peptidoglycan (PGN)+IFN-γ) stimuli or CTO nanoparticles for 24 hours, whereupon OxPhos and glycolytic status was examined using Seahorse Bioscience assays (FIG. 3). Oligomycin nanoparticles (CTO) significantly reduced macrophage basal and maximal respiratory rates compared to untreated and IL-4 stimulated cells (FIGS. 3, C and D), which coincided with significantly higher glycolytic rates (FIG. 3E). The metabolic profile of CTO treated macrophages was most similar to pro-inflammatory macrophages polarized with IFN-γ+PGN, indicating that oligomycin nanoparticles are capable of re-programming macrophage metabolism.

TABLE 1
Nanoparticle features.

| Abbreviations | Features | Purpose |
|---|---|---|
| C | Cy5 | fluorochrome |
| CT | Cy5/Tuftsin | fluorochrome + targeting |
| CTO | Cy5/Tuftsin/ | fluorochrome + targeting + |
| | Oligomycin | ETC inhibitor |

Tuftsin-Conjugated Nanoparticles are Preferentially Internalized by Monocytes During S. aureus PJI

To determine whether tuftsin-conjugated nanoparticles would be targeted to monocytes in vivo during S. aureus PJI, mice received a single intra-articular injection of C or CT nanoparticles at day 7 post-infection. Mice were sacrificed at 1, 2, or 3 days post-injection to evaluate Cy5 signal stability (FIG. 4A). IVIS imaging demonstrated nanoparticle retention in the joint at day 3 post-injection with signals detected out to day 21, indicating that nanoparticles may act as a depot for continued action (FIG. 4B and FIG. 4E). Flow cytometry revealed that >85% of all Cy5⁺ cells were monocytes (CD11b^{high}Ly6G⁻Ly6C⁺F4/80⁻), whereas minimal uptake was detected in MDSCs or neutrophils (FIG. 4C). Macrophages are a minor infiltrate in the PJI model (i.e. <5%); therefore, uptake in biofilm-associated macrophages was negligible. Importantly, uptake of tuftsin-conjugated (CT) nanoparticles by biofilm-associated monocytes was significantly higher compared to non-coated (C) nanoparticles, where approximately 15% of biofilm-associated monocytes were Cy5+ 24 hours after CT nanoparticle injection, which steadily declined to <3.5% by 72 hours (FIG. 4D). These data demonstrate that tuftsin-conjugated nanoparticles are internalized by biofilm-associated monocytes with a high degree of specificity.

### Oligomycin-Containing Nanoparticles Re-Program Biofilm-Associated Monocytes Towards a Pro-Inflammatory Phenotype In Vivo

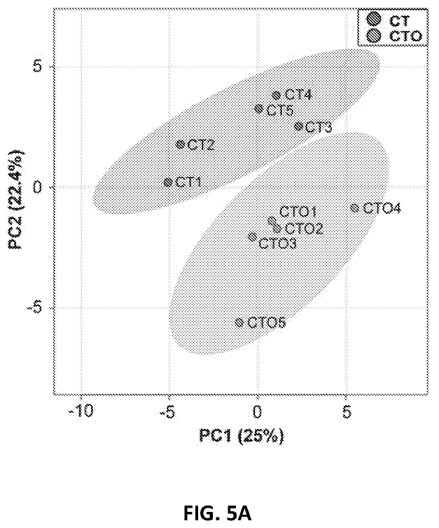

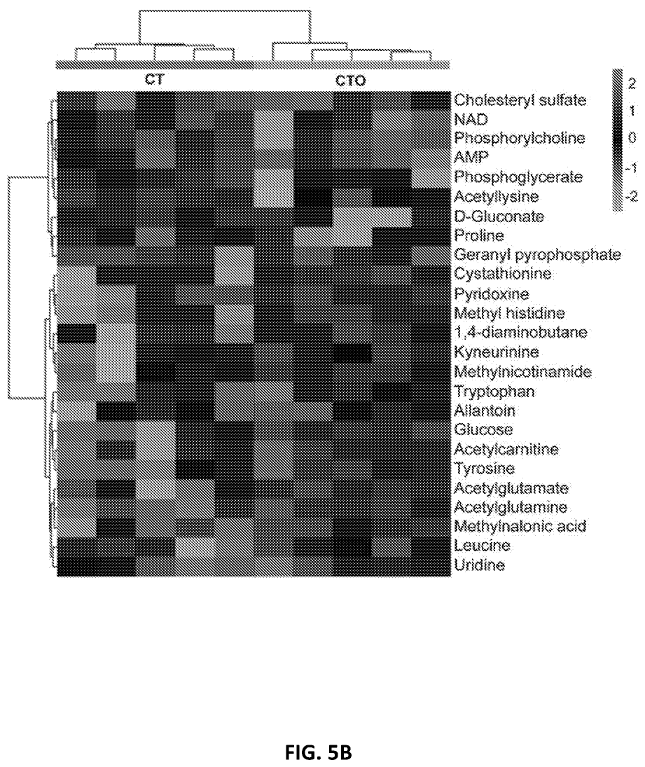

To determine whether the selective uptake of CTO nanoparticles by monocytes in vivo translated into metabolic re-programming, metabolomics was performed. For these studies, mice received a single intra-articular injection of CT or CTO nanoparticles at day 7 post-infection and were sacrificed 3 days later, whereupon polar metabolites were isolated from FACS-purified monocytes (CD11b^{high}Ly6C⁺Ly6G⁻ F4/80⁻) and analyzed by HPLC/MS/MS. All biofilm-associated monocytes were analyzed, since Cy5⁺ monocytes were less frequent at 3 days following nanoparticle injection (FIG. 4D). This interval was selected since studies determined that CTO nanoparticles had no impact on bacterial burdens in the soft tissue, knee joint, femur, or implant 3 days after injection (FIG. 4F). Furthermore, this preceded the significant reductions in bacterial burdens that were observed beginning at 7 days following CTO nanoparticle treatment (FIG. 4F), ensuring that any differences in monocyte activation reflected effects of CTO nanoparticles rather than differences in bacterial burdens, which was significantly altered by day 7. All biofilm-associated monocytes were analyzed, since Cy5+ monocytes were less frequent at 3 days following nanoparticle injection. Principle component analysis (PCA) using unsupervised hierarchical clustering revealed distinct metabolic patterns of monocytes recovered from CT and CTO treated mice (FIGS. 5A and B). Pathway impact analysis of differentially expressed metabolites revealed that arginine, proline, and branched chain amino acid metabolism were most significantly impacted (Table 2). Despite these significant pathway changes, analysis of individual metabolites only identified a few significant differences, most notably decreased NAD⁺ levels in monocytes recovered from CTO treated animals (Table 3), indicative of OxPhos inhibition by oligomycin.

The lack of a clearly defined glycolytic metabolite profile in monocytes following oligomycin nanoparticle treatment was not unexpected, since the signals that dictate cell polarization in vivo are complex compared to in vitro studies where conditions are tightly controlled (Wynn, et al. (2013) Nature 496(7446):445-55). This dichotomy between the monocyte metabolome in vivo and in vitro is reminiscent of the inability of the M1 vs. M2 macrophage polarization state to accurately depict cellular activation states in vivo, since macrophages are highly plastic and often express both pro- and anti-inflammatory markers (Martinez, et al. (2014) F1000prime Rep., 6:13; Nahrendorf, et al. (2016) Cir. Res., 119(3):414-7), which is supportive of the metabolomics data.

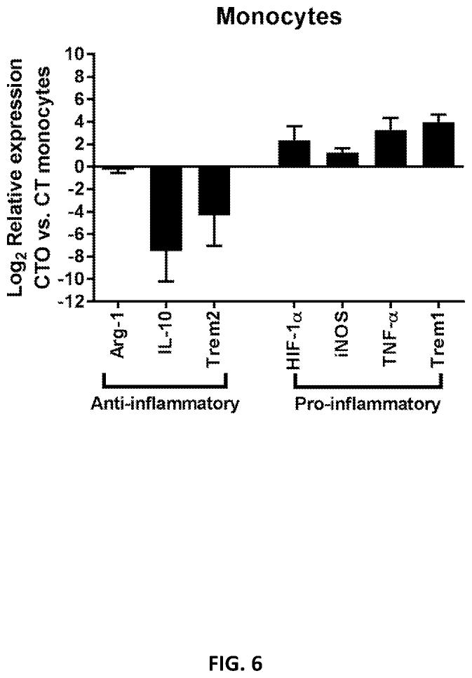

To examine whether the shift in monocyte metabolism in response to oligomycin translated into enhanced pro-inflammatory properties, RT-qPCR was performed on FACS purified monocytes using a panel of pro- and anti-inflammatory genes. Monocytes isolated from CTO treated mice displayed increased pro-inflammatory (HIF-1α, iNOS, TNF-α, and Trem1) concomitant with reduced anti-inflammatory (arginase-1, IL-10, and Trem2) mediators compared to monocytes exposed to control (CT) nanoparticles (FIG. 6). The changes in metabolites and inflammatory mediator expression elicited by oligomycin nanoparticles were likely modest because pₜ of recovered monocytes were Cy5⁺ (FIG. 4D), which further emphasizes the significance of these findings. Earlier intervals following nanoparticle injection were not examined to allow sufficient time for oligomycin to metabolically re-program monocyte metabolism following nanoparticle uptake. Collectively, these results establish that targeting oligomycin to monocytes using tuftsin nanoparticles induces a metabolic shift that promotes their pro-inflammatory properties.

TABLE 2
Identification of the most significantly altered pathways in
monocytes isolated from CTO treated animals as determined
by pathway impact analysis.

| CTO regulated pathways in monocytes | Trend | p-value |
|---|---|---|
| Arginine and Proline Metabolism | ↓ | 0.0134 |
| Valine, Leucine, and Isoleucine Metabolism | ↓ | 0.0113 |

TABLE 3
Intracellular metabolites from CT and CTO treated MDSCs and
monocytes. Fold-change values are expressed as CTO relative to CT.

| | MDSCs | | | |
|---|---|---|---|---|
| | CT | CTO | Fold Δ | p-value |
| NADH | 230.96 | 987.24 | 4.274562 | 0.000108 |
| AMP | 28448.06 | 30887.49 | 1.085751 | 0.473215 |
| phosphorylcholine | 54710.35 | 42805.98 | 0.782411 | 0.088745 |
| phosphoglycerate | 3675.21 | 2552.41 | 0.694494 | 0.008722 |
| amino octanoic acid | 359348.45 | 214208.79 | 0.596103 | 0.014962 |
| octulose monophosphate | 1251.47 | 652.06 | 0.521033 | 0.007184 |
| hexose phosphate | 4513.27 | 1978.78 | 0.438435 | 0.005968 |
| methyl-oxo-pentanoate | 4295.21 | 1879.28 | 0.437530 | 0.114006 |
| proline | 14840.42 | 6255.23 | 0.421499 | 0.021929 |
| argininosuccinate | 13556.30 | 5689.39 | 0.419686 | 0.022393 |
| acetylcarnitine | 4737020.76 | 1980968.73 | 0.418189 | 0.007412 |
| glycerophosphocholine | 224161.08 | 92877.93 | 0.414336 | 0.000115 |
| malate | 6270.01 | 2564.13 | 0.408952 | 0.000511 |
| biotin | 22832.65 | 8173.01 | 0.357953 | 0.001574 |
| carbamoyl aspartate | 15839.22 | 5539.75 | 0.349749 | 0.000060 |
| 1,4-diaminobutane | 1669.81 | 575.51 | 0.344659 | 0.008614 |
| aspartate | 43571.45 | 14625.59 | 0.335669 | 0.004057 |
| acetyl ornithine | 427325.21 | 141033.61 | 0.330038 | 0.006099 |
| arginine | 654825.55 | 215629.05 | 0.329292 | 0.008879 |
| geranyl pyrophosphate | 4059.84 | 1297.46 | 0.319584 | 0.007627 |
| thiamine | 12741.25 | 3979.46 | 0.312328 | 0.000310 |
| phenyl acetylglutamine | 47370.51 | 14792.02 | 0.312262 | 0.006011 |
| pipecolic acid | 966831.86 | 300066.48 | 0.310361 | 0.000021 |
| inosine | 4503.24 | 1373.42 | 0.304984 | 0.001033 |
| methylnicotinamide | 15618.70 | 4716.50 | 0.301978 | 0.017516 |
| methionine sulfoxide | 14441.79 | 4322.62 | 0.299313 | 0.028677 |
| acetylserine | 304852.03 | 90174.14 | 0.295796 | 0.000005 |
| dimethyglycine | 596357.10 | 176288.74 | 0.295609 | 0.000250 |
| mesaconic acid | 3138832.39 | 902198.18 | 0.287431 | 0.002900 |
| itaconic acid | 418700.34 | 120019.94 | 0.286649 | 0.002826 |
| pyrophosphate | 344093.90 | 97693.23 | 0.283914 | 0.002108 |
| leucine | 595958.48 | 168025.76 | 0.281942 | 0.000436 |
| spermidine | 1068.74 | 297.65 | 0.278504 | 0.021936 |
| glutamine | 46272.09 | 12879.45 | 0.278342 | 0.000893 |
| metanephrine | 17549.15 | 4814.09 | 0.274320 | 0.002201 |
| betaine | 46961.12 | 12616.09 | 0.268650 | 0.004982 |
| 7-methylguanosine | 17752.88 | 4763.33 | 0.268313 | 0.000002 |
| citrate | 1792491.13 | 472481.58 | 0.263589 | 0.003571 |
| creatinine | 858105.55 | 224150.40 | 0.261215 | 0.000015 |
| lysine | 412700.45 | 106913.54 | 0.259058 | 0.000155 |
| indole | 463265.15 | 116725.17 | 0.251962 | 0.000222 |
| hypoxanthine | 324937.56 | 80617.09 | 0.248100 | 0.000007 |
| carnitine | 143660.50 | 35568.69 | 0.247588 | 0.007829 |
| lactate | 10938.99 | 2690.17 | 0.245925 | 0.000599 |
| ornithine | 145540.65 | 35614.43 | 0.244704 | 0.000066 |
| choline | 2217322.04 | 531963.86 | 0.239913 | 0.005514 |
| amino isobutyrate | 12290.65 | 2921.84 | 0.237728 | 0.000159 |
| acetyl glutamate | 61562.01 | 14549.64 | 0.236341 | 0.000095 |
| phenylpropiolic acid | 890.53 | 198.35 | 0.222727 | 0.001193 |
| cystathionine | 3953.59 | 876.94 | 0.221810 | 0.040094 |
| glutamate | 1222459.00 | 269634.98 | 0.220568 | 0.005554 |
| 2-hydroxygluterate | 1178.11 | 258.93 | 0.219789 | 0.029155 |
| creatine | 2184525.87 | 477143.44 | 0.218420 | 0.007520 |
| betaine aldehyde | 11707.97 | 2537.86 | 0.216764 | 0.010410 |
| cholesteryl sulfate | 17872.15 | 3857.99 | 0.215866 | 0.011664 |
| anthranil ate | 3867.51 | 824.43 | 0.213167 | 0.012390 |
| methyl histidine | 117630.38 | 24551.32 | 0.208716 | 0.009683 |
| succinate | 5187.26 | 1071.83 | 0.206628 | 0.005598 |
| aconitate | 3470.42 | 701.06 | 0.202011 | 0.001143 |
| pyridoxine | 892902.77 | 171808.98 | 0.192416 | 0.008112 |
| tyrosine | 151214.76 | 29057.79 | 0.192162 | 0.007009 |
| 1-methyladenosine | 31864.27 | 6109.45 | 0.191734 | 0.000007 |
| methylcysteine | 162442.78 | 30426.18 | 0.187304 | 0.007078 |
| tryptophan | 179625.10 | 33535.58 | 0.186698 | 0.004582 |
| glucose | 42435.23 | 7658.38 | 0.180472 | 0.002807 |
| Ng,NG-dimethyl-L-arginine | 311055.15 | 56018.05 | 0.180090 | 0.004851 |
| D-gluconate | 5531.46 | 977.45 | 0.176708 | 0.003715 |
| acetylglutamine | 1991829.48 | 351228.24 | 0.176334 | 0.006212 |
| myo-inositol | 82324.23 | 14296.03 | 0.173655 | 0.005443 |
| xanthosine | 55213.12 | 9201.99 | 0.166663 | 0.000041 |
| allantoin | 8097.74 | 1299.27 | 0.160449 | 0.000602 |
| valine | 30293.38 | 4833.61 | 0.159560 | 0.005609 |
| kyneurinine | 12216.08 | 1884.32 | 0.154249 | 0.005376 |
| nicotinamide | 148691.12 | 22747.25 | 0.152983 | 0.001262 |
| pantothenate | 103332.69 | 15649.02 | 0.151443 | 0.009324 |
| methylmalonic acid | 8013.86 | 1210.10 | 0.151000 | 0.010393 |
| acetyllysine | 10319.45 | 1532.13 | 0.148470 | 0.009164 |
| cytidine | 300405.65 | 43954.29 | 0.146316 | 0.008228 |
| xanthurenic acid | 3490.77 | 490.47 | 0.140504 | 0.012470 |
| hypoxanthine | 3681.43 | 448.85 | 0.121924 | 0.018535 |
| 2-dehydro-D-gluconate | 3741.68 | 417.12 | 0.111478 | 0.008752 |
| beta hydroxybutyrate | 993.14 | 44.63 | 0.044939 | 0.015672 |
| | Monocytes | | | |
| NADH | | | | |
| AMP | 8049.87 | 3738.80 | 0.464454 | 0.013078 |
| phosphorylcholine | 59220.23 | 23283.73 | 0.393172 | 0.003895 |
| phosphoglycerate | 2094.56 | 693.79 | 0.331234 | 0.022996 |
| amino octanoic acid | 198258.16 | 166393.41 | 0.839276 | 0.567904 |
| octulose monophosphate | 1056.36 | 1375.83 | 1.302424 | 0.173201 |
| hexose phosphate | 1261.01 | 1041.04 | 0.825563 | 0.535820 |
| methyl-oxo-pentanoate | 3283.94 | 1856.03 | 0.565183 | 0.049309 |
| proline | 36241.89 | 17299.73 | 0.477341 | 0.059976 |
| argininosuccinate | 13053.01 | 9895.81 | 0.758124 | 0.378851 |
| acetylcarnitine | 2705159.57 | 2617356.63 | 0.967542 | 0.910819 |
| glycerophosphocholine | 284310.52 | 258921.43 | 0.910699 | 0.730178 |
| malate | 6923.16 | 7305.33 | 1.055202 | 0.824362 |
| biotin | 38784.23 | 25758.81 | 0.664157 | 0.210080 |
| carbamoyl aspartate | 17379.64 | 11672.62 | 0.671626 | 0.208280 |
| 1,4-diaminobutane | 1991.87 | 2058.02 | 1.033214 | 0.924452 |
| aspartate | 39461.81 | 30715.02 | 0.778348 | 0.370345 |
| acetyl ornithine | 369919.76 | 329268.98 | 0.890109 | 0.679170 |
| arginine | 537270.27 | 496236.71 | 0.923626 | 0.761768 |
| geranyl pyrophosphate | 3787.62 | 2600.52 | 0.686585 | 0.268201 |
| thiamine | 18692.29 | 13920.87 | 0.744738 | 0.282134 |
| phenyl acetylglutamine | 44993.63 | 30845.69 | 0.685557 | 0.301475 |
| pipecolic acid | 1346786.74 | 1164982.29 | 0.865009 | 0.599083 |
| inosine | 2542.28 | 1834.50 | 0.721595 | 0.392853 |
| methylnicotinamide | 11210.60 | 10691.12 | 0.953661 | 0.872916 |
| methionine sulfoxide | 16613.50 | 16020.02 | 0.964277 | 0.905437 |
| acetylserine | 420870.73 | 361506.78 | 0.858950 | 0.613107 |
| dimethyglycine | 680422.89 | 576081.09 | 0.846652 | 0.545541 |
| mesaconic acid | 2613194.80 | 2171897.97 | 0.831127 | 0.474428 |
| itaconic acid | 377928.56 | 302611.59 | 0.800711 | 0.395602 |
| pyrophosphate | 234661.09 | 211191.49 | 0.899985 | 0.690683 |
| leucine | 681946.44 | 619007.47 | 0.907707 | 0.727735 |
| spermidine | 2486.46 | 1862.20 | 0.748937 | 0.396051 |
| glutamine | 56231.97 | 47633.94 | 0.847097 | 0.601241 |
| metanephrine | 19088.25 | 17776.91 | 0.931301 | 0.825871 |
| betaine | 28747.46 | 23328.76 | 0.811507 | 0.531613 |
| 7-methylguanosine | 27813.06 | 24382.54 | 0.876658 | 0.640393 |
| citrate | 1282441.02 | 1265529.03 | 0.986813 | 0.959020 |
| creatinine | 1283060.49 | 1050524.89 | 0.818765 | 0.502218 |
| lysine | 515826.83 | 448887.04 | 0.870228 | 0.608485 |
| indole | 470724.70 | 339231.95 | 0.720659 | 0.331652 |
| hypoxanthine | 487605.85 | 391813.14 | 0.803545 | 0.441956 |
| carnitine | 206066.04 | 133416.41 | 0.647445 | 0.197903 |
| lactate | 16907.24 | 12507.33 | 0.739762 | 0.256613 |
| ornithine | 137901.12 | 127836.07 | 0.927013 | 0.791126 |
| choline | 2020691.48 | 1739095.78 | 0.860644 | 0.584703 |
| amino isobutyrate | 10622.32 | 9508.84 | 0.895175 | 0.646089 |
| acetyl glutamate | 65764.10 | 70330.71 | 1.069439 | 0.806158 |
| phenylpropiolic acid | 816.78 | 786.69 | 0.963151 | 0.939851 |
| cystathionine | 2903.78 | 2815.83 | 0.969714 | 0.904532 |
| glutamate | 1260582.55 | 1048365.28 | 0.831651 | 0.515478 |
| 2-hydroxygluterate | 551.86 | 684.59 | 1.240517 | 0.571860 |
| creatine | 1924037.06 | 1500330.74 | 0.779783 | 0.351090 |
| betaine aldehyde | 9938.89 | 8862.38 | 0.891687 | 0.744600 |
| cholesteryl sulfate | 18794.03 | 12592.39 | 0.670021 | 0.253899 |
| anthranilate | 3144.91 | 1898.86 | 0.603788 | 0.329324 |
| methyl histidine | 106123.92 | 110072.95 | 1.037211 | 0.892255 |
| succinate | 5581.73 | 4892.55 | 0.876530 | 0.675193 |
| aconitate | 4137.89 | 4142.85 | 1.001199 | 0.997102 |
| pyridoxine | 729019.37 | 778520.79 | 1.067901 | 0.803706 |
| tyrosine | 130431.15 | 123118.45 | 0.943934 | 0.847570 |
| 1-methyladenosine | 37126.74 | 33451.71 | 0.901014 | 0.763139 |
| methylcysteine | 141086.24 | 127112.91 | 0.900959 | 0.730352 |
| tryptophan | 133199.94 | 137709.31 | 1.033854 | 0.920956 |
| glucose | 33657.50 | 32159.00 | 0.955478 | 0.882474 |
| Ng,NG-dimethyl-L-arginine | 305985.17 | 265305.75 | 0.867054 | 0.623452 |
| D-gluconate | 5885.43 | 4126.70 | 0.701172 | 0.318103 |
| acetylglutamine | 1653195.62 | 1708981.37 | 1.033744 | 0.904038 |
| myo-inositol | 63890.10 | 59667.70 | 0.933911 | 0.799933 |
| xanthosine | 74332.18 | 65463.52 | 0.880689 | 0.702918 |
| allantoin | 7292.10 | 8242.82 | 1.130377 | 0.708690 |
| valine | 31134.19 | 26913.39 | 0.864432 | 0.607698 |
| kyneurinine | 7923.02 | 10551.98 | 1.331812 | 0.431999 |
| nicotinamide | 92465.86 | 106831.39 | 1.155360 | 0.732730 |
| pantothenate | 94030.70 | 86732.95 | 0.922390 | 0.799106 |
| methylmalonic acid | 5162.45 | 6284.79 | 1.217403 | 0.507959 |
| acetyllysine | 11230.10 | 7776.12 | 0.692436 | 0.237901 |
| cytidine | 221483.90 | 166574.21 | 0.752083 | 0.384881 |
| xanthurenic acid | 4869.40 | 5399.37 | 1.108837 | 0.760682 |
| hypoxanthine | 4291.35 | 3793.19 | 0.883915 | 0.697541 |
| 2-dehydro-D-gluconate | 3126.74 | 2136.40 | 0.683268 | 0.368494 |
| beta hydroxybutyrate | 550.54 | 375.74 | 0.682504 | 0.502109 |

### Monocyte Targeted Oligomycin Nanoparticles Induce Metabolomic and Gene Expression Changes in MDSCs, Indicative of Cellular Crosstalk

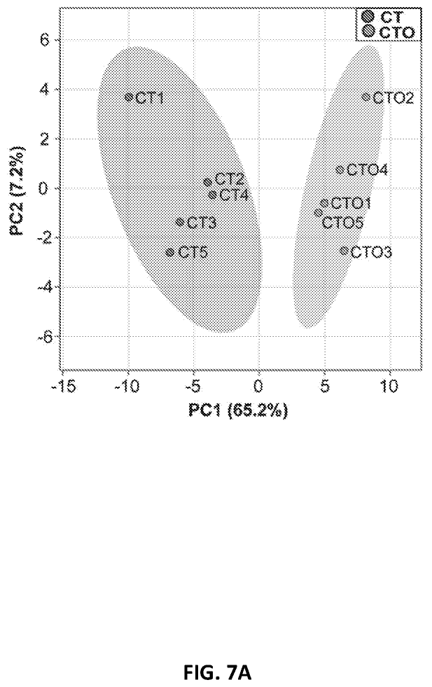

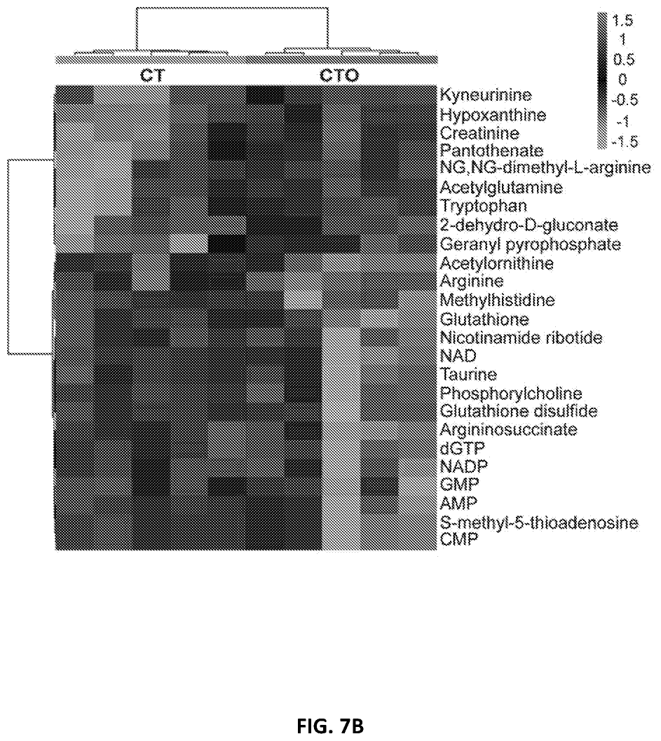

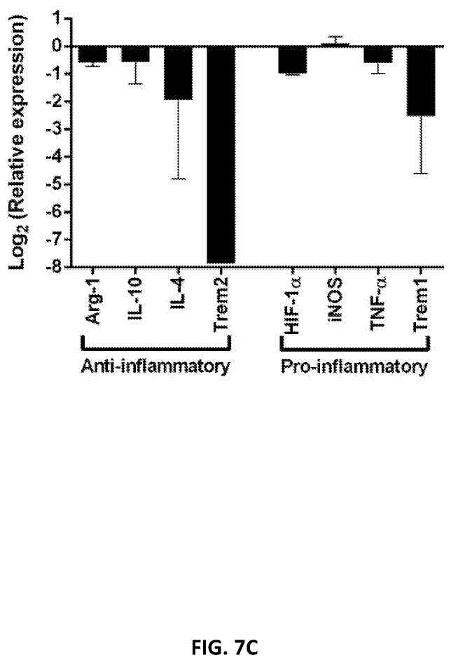

MDSCs have a significant role in promoting monocyte anti-inflammatory activity during S. aureus orthopedic biofilm infection (Heim, et al. (2015) J. Leukoc. Biol., 98:1003-1013; Heim, et al. (2014) J. Immunol., 192:3778-3792; Heim, et al. (2015) J. Immunol., 194:3861-3872). Although <0.01% of MDSCs internalized tufstin nanoparticles (FIG. 4C), the degree of cross-talk between metabolically altered monocytes and MDSCs was determined. MDSCs (CD11b^{high}Ly6C⁺Ly6G⁺F4/80⁻) were purified from CT and CTO treated animals, whereupon metabolomics and inflammatory gene expression were examined as was performed for monocytes. Remarkably, although nanoparticle uptake was negligible in MDSCs, their metabolic profile was dramatically affected by CTO treatment (FIGS. 7A and B), with the number of significant differentially expressed metabolites far outnumbering those for monocytes (Table 3). Metabolic pathway analysis revealed significant increases in glutathione, nicotinamide, taurine, and purine metabolism, whereas arginine, glutamine, and cyclic chain amino acids were significantly reduced (Table 4). In contrast, RT-qPCR analysis was less straightforward, since anti-/pro-inflammatory mediator expression in MDSCs following CTO nanoparticle treatment did not display a discernable gene expression pattern consistent with a specific function (FIG. 7C). These results are the first to demonstrate that metabolic re-programming of monocytes can impact MDSC metabolism, thereby supporting MDSC-monocyte crosstalk.

TABLE 4
Identification of the most significantly altered pathways in MDSCs isolated
from CTO treated animals as determined by pathway impact analysis.

| CTO regulated pathways in monocytes | Trend | p-value |
|---|---|---|
| Glutathione Metabolism | ↑ | 0.000024 |
| Arginine and Proline Metabolism | ↓ | 0.00004 |
| Nicotinamide and Nicotinate Metabolism | ↑ | 0.000054 |
| Taurine and Hypotaurine Metabolism | ↑ | 0.000267 |
| Alanine, Aspartate and Glutamate Metabolism | ↓ | 0.000675 |
| Phenylalanine, Tyrosine, and Tryptophan Metabolism | ↓ | 0.00761 |
| Glutamine and Glutamate Metabolism | ↓ | 0.00747 |

Nanoparticle-Mediated Delivery of Oligomycin to Monocytes Attenuates Established S. aureus PJI

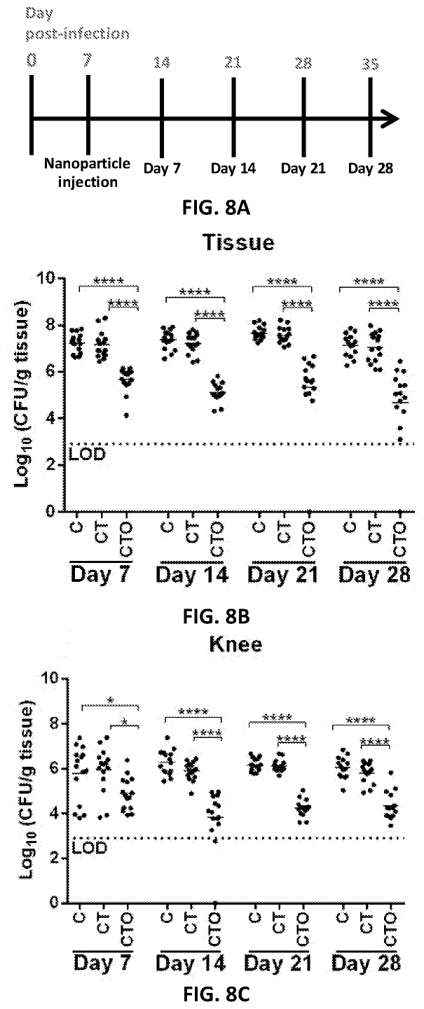

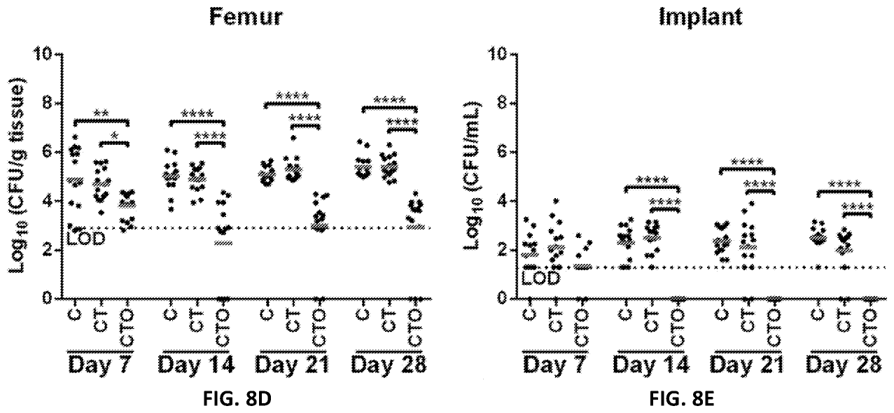

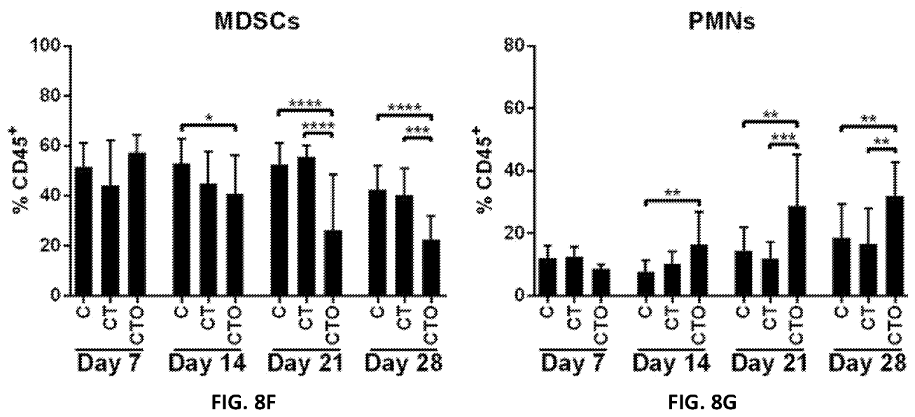

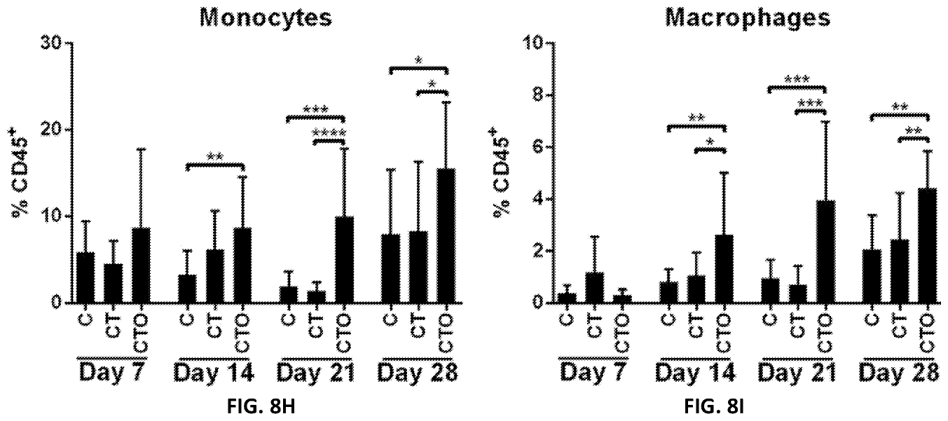

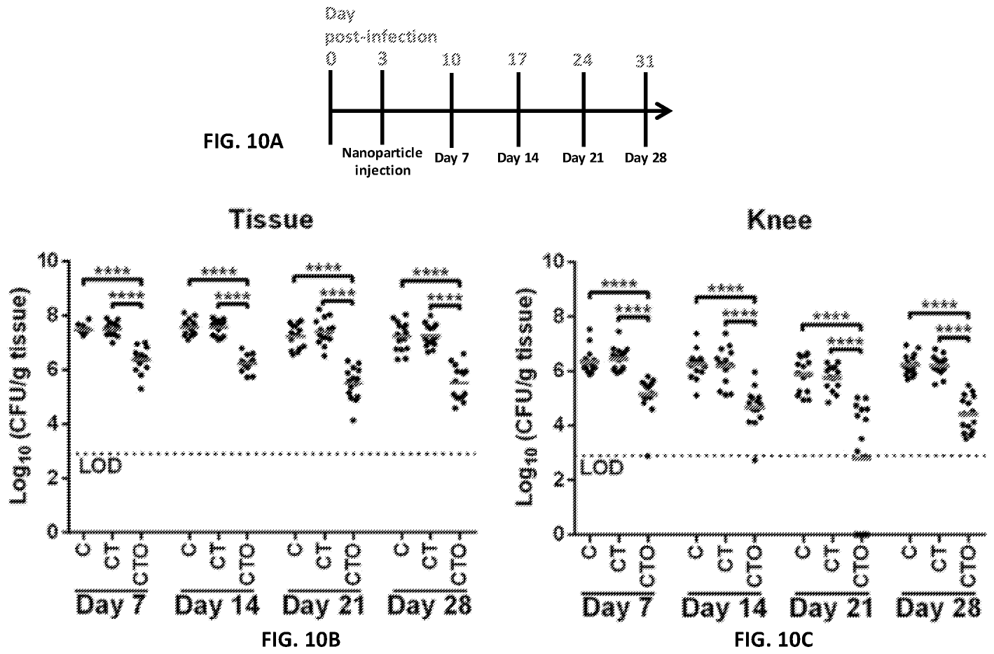

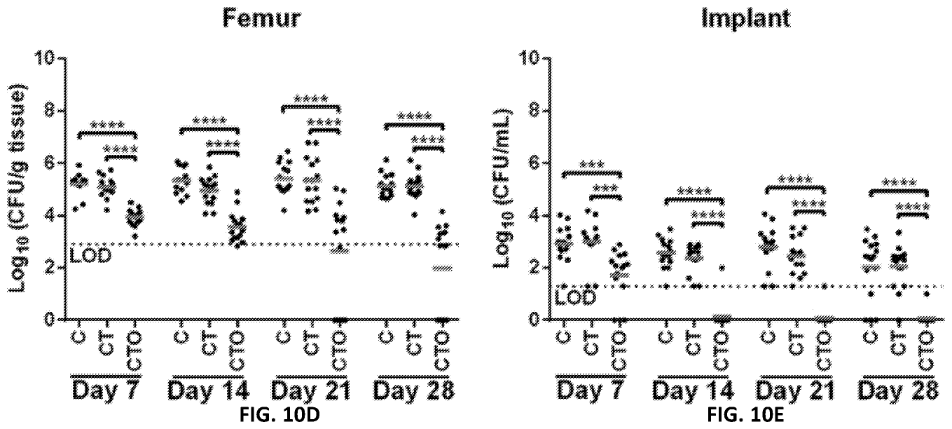

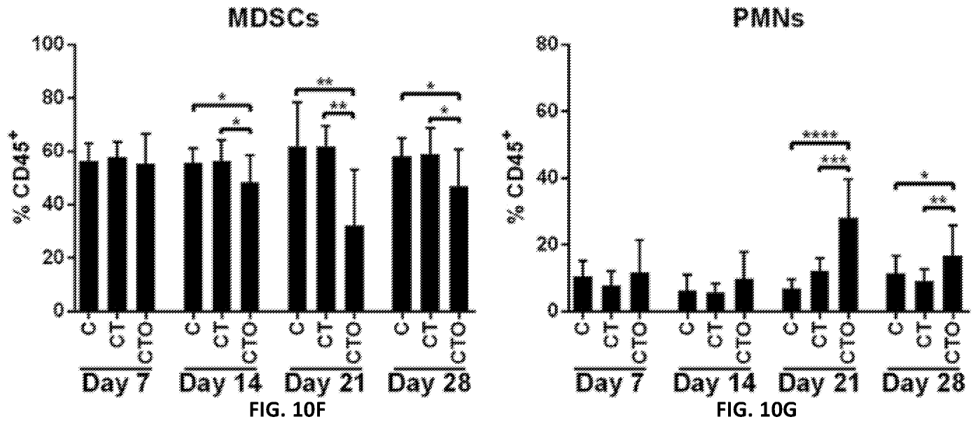

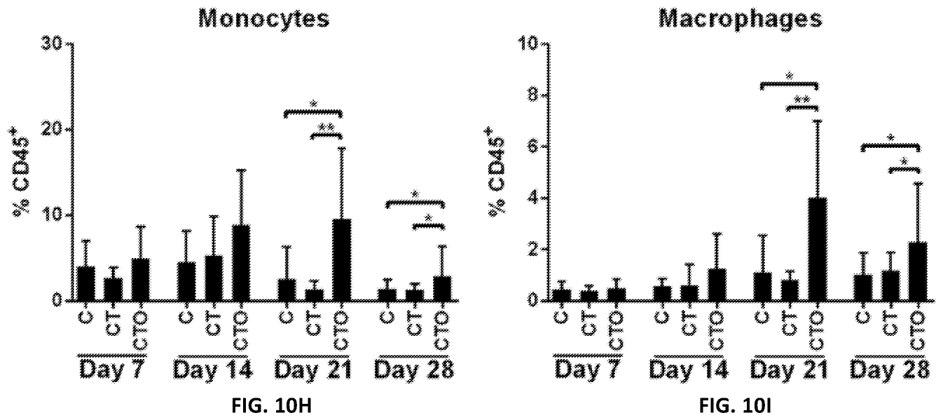

It was then determined whether the observed metabolic re-programming and pro-inflammatory properties of biofilm-associated monocytes following oligomycin nanoparticle administration would impact biofilm clearance during S. aureus PJI. Two treatment paradigms were designed to examine the effect of monocyte metabolic re-programming during biofilm development versus an established biofilm (days 3 and 7 post-infection, respectively). Mice received a single intra-articular injection of control (C and CT) or oligomycin (CTO) nanoparticles on either day 3 (FIG. 10) or day 7 post-infection (FIG. 8), whereupon bacterial burdens and leukocyte infiltrates were examined over a 28 day period. In both settings, bacterial burdens were significantly reduced in the soft tissue (FIG. 10B and FIG. 8B), joint (FIG. 10C and FIG. 8C), femur (FIG. 10D and FIG. 8D), and implant (FIG. 10E and FIG. 8E) between 7 and 28 days following nanoparticle injection. Interestingly, despite the reductions in bacterial titers at day 7 with CTO nanoparticles in both treatment paradigms, no significant differences in MDSC (CD11b^{high}Ly6C⁺Ly6G⁺F4/80⁻) (FIG. 10F and FIG. 8F), PMN (CD11b^{low}Ly6C⁺Ly6G⁺F4/80⁻) (FIG. 10G and FIG. 8G), monocyte (CD11b^{high}Ly6C⁺Ly6G⁻F4/80⁻) (FIG. 10H and FIG. 8H), or macrophage (CD11b^{high}Ly6C⁻Ly6G⁻F4/80⁺) (FIG. 10I and FIG. 8I) infiltrates were observed at day 7. However, at later time points (i.e. days 14 to 28 after nanoparticle treatment) mice receiving CTO nanoparticles had significantly fewer MDSCs concomitant with increased PMN, monocyte, and macrophage infiltrates, reflective of a more pro-inflammatory milieu (FIG. 8 and FIG. 10). Therefore, the observed reductions in bacterial burdens at day 7 may result from enhanced bactericidal activity of biofilm-associated monocytes following oligomycin treatment, since cells exhibited increased pro-inflammatory mediator expression that typically coincides with enhanced ROS production (West, et al. (2011) Nature 472(7344):476-80; Yang, et al. (2009) J. Immunol., 182(6):3696-705).

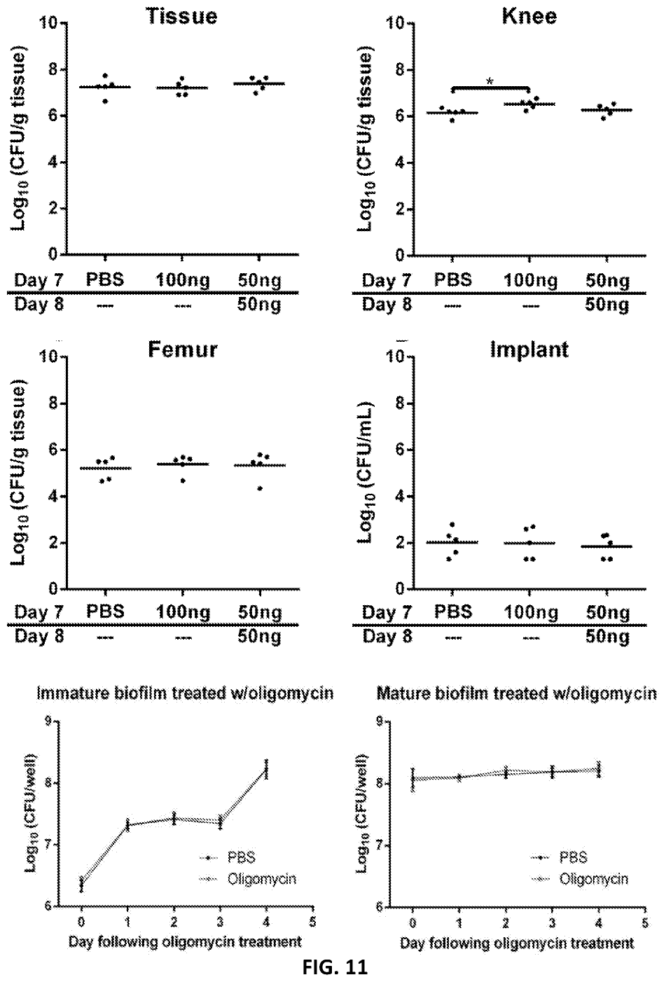

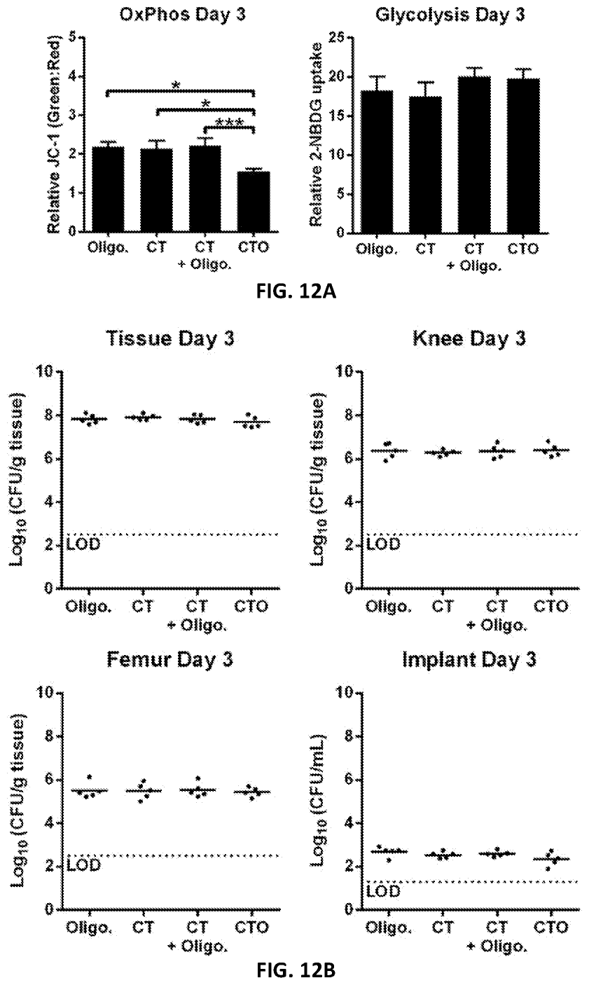

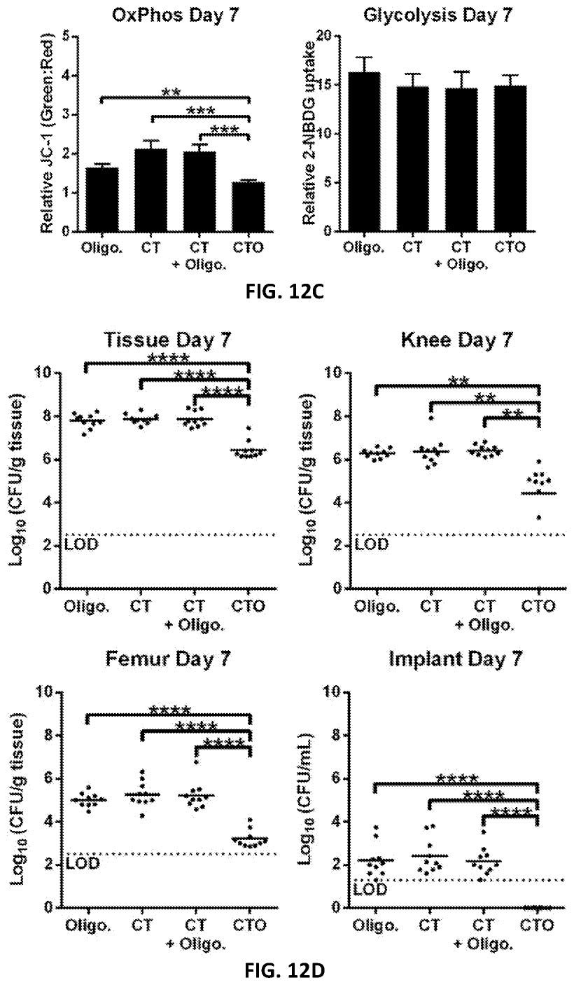

To confirm that the ability of oligomycin nanoparticles to attenuate S. aureus biofilm burdens resulted from monocyte metabolic reprogramming rather than direct antibacterial activity, mice were treated with free oligomycin only, empty nanoparticles (CT) with free oligomycin (not loaded), or oligomycin loaded nanoparticles (CTO). Free oligomycin was administered at a dose that was equivalent to the nanoparticle formulation (100 ng). Only CTO nanoparticles led to significant reductions in monocyte OxPhos activity, as revealed by JC-1 staining (FIGS. 12A and 12C). This was independent of bacterial burdens, since CTO nanoparticles reduced monocyte OxPhos as early as day 3 post-treatment when no differences in biofilm titers were observed (FIG. 12B). Overall, no changes in glycolytic activity were detected with CTO nanoparticles (FIGS. 12A and 12C). However, the reductions in OxPhos support a net glycolytic bias in monocytes following CTO treatment, which agrees with their enhanced pro-inflammatory activity (FIG. 6). Importantly, no changes in monocyte OxPhos or biofilm burdens were observed in animals treated with free oligomycin or CT nanoparticles+free oligomycin (not loaded) at either days 3 or 7 after intra-articular injection (FIG. 12). Furthermore, oligomycin delivery by direct intra-articular injection into the infected joint in either one bolus or two sequential doses also had no effect on bacterial burdens (FIG. 11). Finally, oligomycin displayed no bactericidal activity against S. aureus during early biofilm formation or mature biofilms in vitro (FIG. 11), in agreement with reports demonstrating that oligomycin alone does not affect S. aureus (Lobritz, et al. (2015) Proc. Natl. Acad. Sci., 112(27):8173-80; Vestergaard, et al. (2017) mBio 8(5): e01114-17). Collectively, these results establish that the anti-biofilm effects of oligomycin are dependent on targeted intracellular delivery to monocytes and their subsequent metabolic reprogramming to elicit pro-inflammatory activity, and not as an antibiotic.

Synergistic Action of Oligomycin Nanoparticles and Systemic Antibiotics to Clear Established S. aureus Biofilm Infection

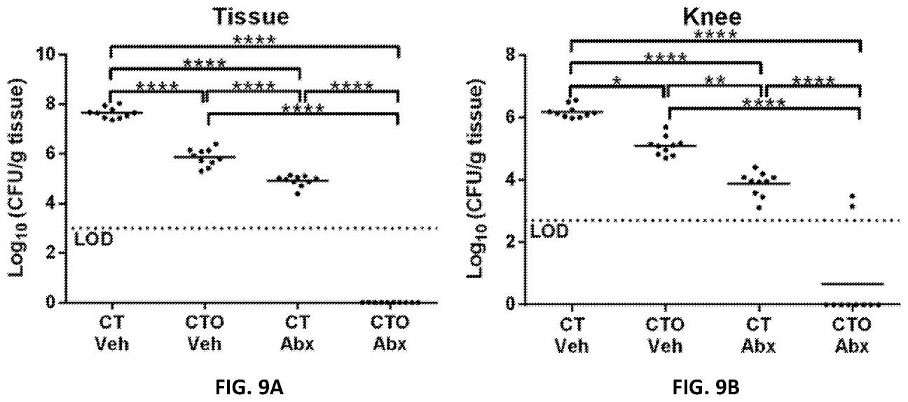

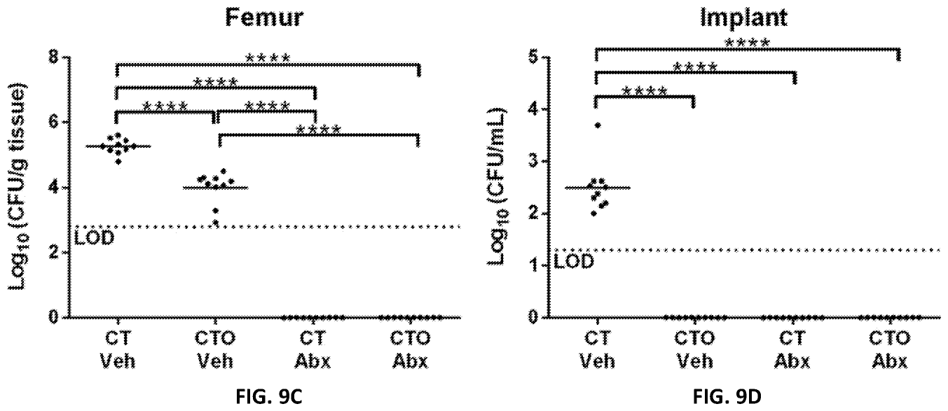

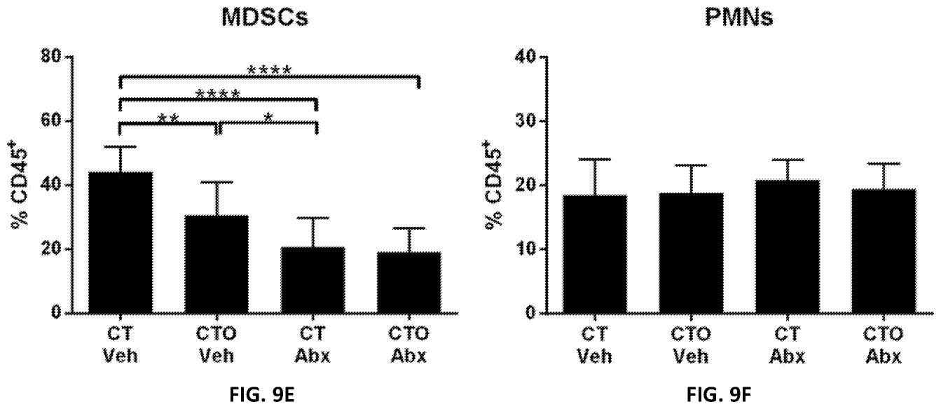

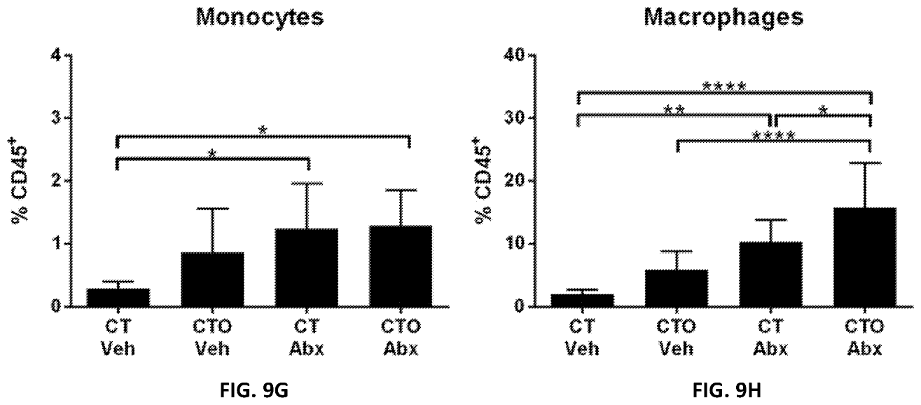

Based on the finding that oligomycin nanoparticles transformed the biofilm milieu by promoting monocyte pro-inflammatory activity and leukocyte recruitment, residual bacteria may have less biofilm and more planktonic characteristics. To address this possibility, mice received CT or CTO nanoparticles on day 7 post-infection, whereupon systemic antibiotics (25 mg/kg/day rifampin and 5 mg/kg/day daptomycin) were administered 7 days later for a one week duration after which mice were sacrificed (corresponding to day 21 post-infection). Remarkably, bacterial burdens were largely below the limit of detection in the soft tissue, joint, femur, and implant of animals that received CTO nanoparticles and antibiotic, whereas bacteria still remained in mice treated with control CT nanoparticles and antibiotics or CTO nanoparticles alone (FIG. 9). These changes were reflected by reduced MDSCs and increased monocyte and macrophage recruitment (FIG. 9, E-H), which the results show are re-programmed towards a pro-inflammatory phenotype by oligomycin delivery (FIGS. 5 and 6). In contrast, bacteria remained in the tissue and knee of mice treated with control CT nanoparticles and antibiotics or CTO nanoparticles alone (FIG. 9, A-B). The ability of systemic antibiotics to clear bacteria in the femur and implant of animals receiving control CT nanoparticles (CT Abx) likely resulted from the high doses of daptomycin and rifampin that were used, which have been shown by others to exhibit some anti-biofilm activity in vivo (Mandell, et al. (2019) J. Orthop. Res., 37(7):1604-9; Raad, et al. (2007) Antimicrob. Agents Chemother., 51(5):1656-60; John, et al. (2009) Antimicrob. Agents Chemother., 53(7):2719-24; Meeker, et al. (2016) Antimicrob. Agents Chemother., 60(10):5688-94). In addition, bacterial burdens in the femur and implant are not as robust compared to the knee joint and surrounding tissue, which may make the former more susceptible to antibiotic-mediated clearance. Indeed, daptomycin (10 mg/kg) reduced S. aureus burdens on orthopedic implants to near the limit of detection (i.e. ˜<10² CFU) in the same mouse PJI model, whereas titers in the joint were less affected (Niska, et al. (2012) PloS One 7(10):e47397). Importantly, daptomycin and rifampin were not capable of clearing infection in the tissue or joint unless combined with oligomycin nanoparticles (FIGS. 9A-9B), demonstrating the need for concurrent monocytic metabolic reprogramming. This is the first demonstration of an approach capable of clearing an established biofilm infection, which was achieved through the combined action of modulating host immunity to increase S. aureus susceptibility to conventional antibiotics.

Approximately one million knee and hip arthroplasties are performed in the United States annually, with PJIs representing the most common complication (Tande, et al. (2014) MBio 5:e01910-01914; Tande, et al. (2014) Clin. Microbiol. Rev., 27:302-345; Kurtz, et al. (2007) J. Bone Joint Surg. Am., 89:780-785; Kurtz, et al. (2007) J. Bone Joint Surg. Am., 89(Suppl 3):144-151; Teterycz, et al. (2010) Intl. J. Inf. Dis., 14:e913-e918; Arduino, et al. (2015) Antimicrob Resist. Infect. Control 4:13; Dapunt, et al. (2016) Materials (Basel) 9:E871). These infections are associated with significant morbidity and medical expenses, since the current standard-of-care requires multiple surgical interventions over a period of months, during which patient mobility is limited (Tande, et al. (2014) MBio 5:e01910-01914; Tande, et al. (2014) Clin. Microbiol. Rev., 27:302-345; Teterycz, et al. (2010) Intl. J. Inf. Dis., 14:e913-e918; Moran, et al. (2010) J. Antimicrob. Chemother., 65(Suppl 3):45-54; Pulido, et al. (2008) Clin. Orthop. Relat. Res., 466:1710-1715). S. aureus is the second most common cause of PJI behind S. epidermidis (Tande, et al. (2014) MBio 5:e01910-01914; Tande, et al. (2014) Clin. Microbiol. Rev., 27:302-345; Teterycz, et al. (2010) Intl. J. Inf. Dis., 14:e913-e918; Arduino, et al. (2015) Antimicrob Resist. Infect. Control 4:13; Dapunt, et al. (2016) Materials (Basel) 9:E871) and biofilm cannot typically be cleared by antibiotics without removing the prosthesis, since the metabolic dormancy of biofilm-associated bacteria decreases their antibiotic susceptibility (Tande, et al. (2014) MBio 5:e01910-01914; Tande, et al. (2014) Clin. Microbiol. Rev., 27:302-345; Moormeier, et al. (2017) Mol. Microbiol., 104:365-376; Moormeier, et al. (2014) MBio 5:e01341-01314). Further compounding this issue is the ability of S. aureus to promote an anti-inflammatory milieu typified by the polarization of anti-inflammatory monocytes, abundance of MDSCs, and paucity of neutrophils and T cells (Hanke, et al. (2013) J. Immunol., 190:2159-2168; Hanke, et al. (2012) Front. Cell Infect. Microbiol., 2:62; Heim, et al. (2015) J. Leukoc. Biol., 98:1003-1013; Heim, et al. (2014) J. Immunol., 192:3778-3792; Heim, et al. (2015) J. Immunol., 194:3861-3872). Monocytes can be a key effector cell during PJI in a permissive microenvironment, such as during MDSC depletion, where their pro-inflammatory activity is augmented leading to reduced biofilm burdens (Hanke, et al. (2013) J. Immunol., 190:2159-2168; Heim, et al. (2015) J. Leukoc. Biol., 98:1003-1013; Heim, et al. (2014) J. Immunol., 192:3778-3792). This study is the first to demonstrate that biofilm-associated monocytes favor OxPhos over glycolysis, which is responsible for skewing cells towards an anti-inflammatory phenotype. This was established by modulating monocyte metabolism with oligomycin using a novel targeted nanoparticle delivery system, which cleared established S. aureus biofilms in combination with systemic antibiotics.

Oligomycin promotes macrophage pro-inflammatory activity by inhibiting ATP synthase and OxPhos (Izquierdo, et al. (2015) J. Immunol., 195:2442-2451). Oligomycin was selected over other metabolic inhibitors because it is less potent and would dampen but not completely block OxPhos, whereas stronger inhibitors, such as rotenone, are extremely toxic and have been shown to inhibit macrophage viability and function (Dietl, et al. (2010) J. Immunol., 184:1200-1209; Sherer, et al. (2003) J. Neurosci., 23:10756-10764; Sherer, et al. (2003) Exp. Neurol., 179:9-16; Yamagishi, et al. (2001) J. Biol. Chem., 276:25096-25100). Furthermore, a nanoparticle-directed method was preferred in vivo because of potential toxicity associated with a non-targeted approach (Kramar, et al. (1984) Agents Actions 15:660-663; Vaamonde-Garcia, et al. (2017) BMC Musculoskelet. Disord., 18:254; Sherer, et al. (2003) J. Neurosci., 23:10756-10764; Sherer, et al. (2003) Exp. Neurol., 179:9-16). In this study, nanoparticles were conjugated with tuftsin to facilitate FcR-mediated uptake in monocytes (Jain, et al. (2012) Biomacromolecules 13:1074-1085; Jain, et al. (2015) Biomaterials 61:162-177; Dutta, et al. (2008) Eur. J. Pharm. Sci., 34:181-189), which are the most abundant mononuclear phagocyte population during S. aureus PJI (Heim, et al. (2015) J. Leukoc. Biol., 98:1003-1013; Heim, et al. (2014) J. Immunol., 192:3778-3792; Heim, et al. (2015) J. Immunol., 194:3861-3872; Heim, et al. (2017) J. Orthop. Res., 36(6):1605-1613). Although other FcR⁺ cells infiltrate S. aureus biofilms, they are either of low abundance (i.e. PMNs) or express less FcR (i.e. MDSCs) compared to monocytes (Bronte, et al. (2016) Nat. Commun., 7:12150).

Importantly, oligomycin nanoparticles were capable of metabolically re-programming biofilm-associated monocytes, which coincided with their polarization to a pro-inflammatory state, typified by increased pro- and reduced anti-inflammatory gene expression. These effects were remarkable given the finding that <5% of monocytes displayed direct evidence of nanoparticle uptake by Cy5-positivity at the time of analysis. However, it is also possible that a greater number of monocytes had internalized nanoparticles but were no longer Cy5⁺, since fluorescence is likely quenched in the phagosome as has been reported for other fluorochromes (Schwartz, et al. (2009) J. Immunol., 183:2632-2641). Metabolic pathway analysis of significantly downregulated metabolites showed that arginine, proline, and branched chain amino acid metabolism were most significantly altered in monocytes isolated from CTO treated mice. Decreased amino acid abundance likely corresponds to increased protein synthesis to satisfy the need for metabolic intermediates during immune activation (O'Neill, L. A. (2015) Immunity 42:393-394; O'Neill, et al. (2016) Nat. Rev. Immunol., 16:553-565; Yoon, et al. (2018) Front. Immunol., 9:53). Citrate and succinate accumulation was expected due to the glycolytic shift induced by oligomycin treatment and block in TCA cycle enzymes; however, both were less abundant in CTO monocytes (O'Neill, et al. (2016) J. Exp. Med., 213:15-23; O'Neill, L. A. (2015) Immunity 42:393-394; O'Neill, et al. (2016) Nat. Rev. Immunol., 16:553-565; Infantino, et al. (2011) Mol. Genet. Metab., 102:378-382). Glucose and glucose-6-phosphate were also decreased in CTO monocytes, which prior in vitro studies showed were increased in pro-inflammatory macrophages (Phan, et al. (2017) Immunity 46:714-729). Nevertheless, secondary metabolites of the TCA cycle, such as aconitate and 2-hydroxyglutarate, were increased in CTO monocytes (Geeraerts, et al. (2017) Front. Immunol., 8:289; Phan, et al. (2017) Immunity 46:714-729; Jha, et al. (2015) Immunity 42:419-430; Cordes, et al. (2015) Annu. Rev. Nutr., 35:451-473; Cordes, et al. (2016) J. Biol. Chem., 291:14274-14284; Intlekofer, et al. (2015) Cell Metab., 22:304-311), indicating an inhibition of the TCA cycle that supports a glycolytic bias. The endogenous macrophage activator uric acid and its metabolite, allantoin, were also more abundant in CTO monocytes (Orlowsky, et al. (2014) BMC Musculoskelet. Disord., 15:318; Shi, et al. (2003) Nature 425:516-521) as were methyl-malonic acid and uridine, consistent with prior in vitro studies of LPS stimulated monocytes with potent pro-inflammatory properties (Fei, et al. (2016) Sci. Rep., 6:22637).

Although these metabolite patterns do not follow the strict definition of glycolytic or OxPhos metabolism that has been described in vitro (Yoon, et al. (2018) Front. Immunol., 9:53; Rattigan, et al. (2018) PLoS One 13:e0194126), it is demonstrated herein that oligomycin induced a metabolic shift in biofilm-associated monocytes in vivo. It is important to recognize that the metabolomic signatures defined for pro- vs. anti-inflammatory macrophages are largely based on in vitro studies, where cells are polarized to the extremes of activation states (Yoon, et al. (2018) Front. Immunol., 9:53; Rattigan, et al. (2018) PLoS One 13:e0194126). In vivo, monocytes/macrophages receive a complex array of signals and exist in a spectrum of activation states, with phenotypic and metabolic plasticity (Biswas, et al. (2010) Nat. Immunol., 11:889-896; Mosser, et al. (2008) Nat. Rev. Immunol., 8:958-969; Sica, et al. (2012) J. Clin. Invest., 122:787-795; Martinez, et al. (2014) F1000 Prime Rep., 6:13; Nahrendorf, et al. (2016) Circ. Res., 119:414-417). Therefore, it is not unexpected that the metabolic profile of biofilm-associated monocytes possessed attributes of both OxPhos and glycolysis, which may be further explained by the milder OxPhos inhibitory activity of oligomycin as described earlier (Izquierdo, et al. (2015) J. Immunol., 195:2442-2451; Jastroch, et al. (2010) Essays Biochem., 47:53-67). Importantly, transformation of biofilm-associated monocytes to a pro-inflammatory state following oligomycin treatment was confirmed by RT-qPCR, where monocytes recovered from CTO treated animals displayed increased pro- and reduced anti-inflammatory gene expression as compared to monocytes from mice receiving CT control nanoparticles. These changes in monocyte metabolism and pro-inflammatory activity were independent of bacterial burden, which was equivalent between animals receiving oligomycin and control nanoparticles at day 3 post-treatment, and increased monocyte pro-inflammatory activity is likely responsible for the reduction in S. aureus titers at day 7 onward. This study is the first demonstration of the utility of nanoparticle-directed metabolic re-programming to modulate monocyte activation in vivo in the context of biofilm-associated infection.

An unexpected finding in this study was that biofilm-associated MDSCs displayed marked alterations in their metabolome, despite that fact that <0.01% cells exhibited evidence of nanoparticle uptake. This is the first demonstration of monocyte-MDSC metabolic crosstalk during biofilm infection, which supports observations where MDSC depletion led to enhanced monocyte pro-inflammatory activity. Metabolic pathway analysis revealed that MDSCs recovered from CTO treated mice had significant increases in glutathione, nicotinamide, taurine, and purine metabolism, whereas arginine, glutamine, negatively charged, and cyclic chain amino acids were down-regulated. It is difficult to predict the impact of these metabolic changes, since few studies have explored MDSC metabolism, especially in vivo. Without speculation as to the mechanism of how MDSC metabolism is altered, this pathway signature would be generally consistent with a glycolytic preference. Similar to monocytes, a decrease in amino acids likely corresponds to an increase in protein synthesis or need for metabolic intermediates (O'Neill, L. A. (2015) Immunity 42:393-394; O'Neill, et al. (2016) Nat. Rev. Immunol., 16:553-565; Yoon, et al. (2018) Front. Immunol., 9:53). An increase in GSH/GSSH has been shown to prime T cells for glycolytic metabolism and Th1 pro-inflammatory activity (Dobashi, et al. (2001) Clin. Exp. Immunol., 124:290-296; Mak, et al. (2017) Immunity 46:675-689). Similarly, the increase in NADH/NAD+(2.489-fold higher in MDSCs recovered from CTO compared to CT treated mice) corresponds to a reduction in electron transport chain activity (Rattigan, et al. (2018) PLoS One 13:e0194126; Rovito, et al. (2013) Br. J. Dermatol., 169(Suppl 2):15-24). While elevated NADH/NAD+ should increase lactic acid fermentation and lactate production, intracellular lactate levels were 4-fold lower in MDSCs from CTO treated animals. One potential explanation to account for this finding is that lactate is exported from the cell by monocarboxylate transporters (MCTs), which are up-regulated following immune activation (Geeraerts, et al. (2017) Front. Immunol., 8:289). These results are the first to demonstrate that metabolic re-programming of monocytes can impact MDSC metabolism in any disease model.

Major challenges pertaining to the treatment of PJI are the inability to clear infection without removing the prosthesis, and prevention of infection recurrence. Based on the findings that oligomycin nanoparticles augmented monocyte pro-inflammatory activity, it was examined how this impacted biofilm burden. Remarkably, S. aureus titers were significantly reduced in mice receiving oligomycin nanoparticles out to 28 days post-injection. This was accompanied with a reduction in MDSCs concomitant with increased monocyte, PMN, and macrophage infiltrates, which were likely responsible for biofilm clearance. This relationship is similar to the observations where reductions in MDSCs lead to increased monocyte, PMN, and/or macrophage influx and decreased biofilm burdens. These findings are remarkable for several reasons. First, only a single injection of CTO nanoparticles was required demonstrating sustained action, which is a desirable feature for therapeutic interventions. Second, nanoparticle treatment was not initiated until biofilm infection was established (i.e. day 7 post-infection), which represents the most challenging treatment scenario. Third, combined treatment with CTO nanoparticles and systemic antibiotics reduced infectious burden to below the limit of detection. The shift in monocyte pro-inflammatory activity by oligomycin promotes biofilm disruption and transforms metabolically dormant biofilm-associated bacteria into a planktonic mode of growth, which is more susceptible to antibiotic action. This is the first demonstration of an approach that is capable of clearing established biofilms without removal of the infected implant.

Understanding the complex and dynamic interactions between S. aureus biofilm and the host immune response is important for developing a novel therapeutic approach to reduce the morbidity associated with PJI. This study is the first to demonstrate that biofilm-associated monocytes experience a metabolic shift to favor OxPhos that promotes their anti-inflammatory activity and that cells can be metabolically re-programmed using a novel nanoparticle targeted approach to promote their pro-inflammatory properties, which results in biofilm clearance.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A micelle comprising
a) a block copolymer comprising an ionically charged polymeric segment and a non-ionically charged polymeric segment, wherein said ionically charged polymeric segment is linked to hydrophobic moieties and forms the core of the micelle, and wherein said non-ionically charged polymeric segment is hydrophilic;
b) a leukocyte specific targeting moiety, wherein said targeting moiety is linked to said non-ionically charged polymeric segment; and
c) an inhibitor of oxidative phosphorylation, wherein said inhibitor is within the core of the micelle.

2: The micelle of claim 1, wherein said ionically charged polymeric segment is cross-linked.

3: The micelle of claim 1, wherein said targeting moiety targets or binds to monocytes and macrophages.

4: The micelle of claim 1, wherein said non-ionically charged polymeric segment comprises polyethylene oxide.

5: The micelle of claim 1, wherein said ionically charged polymeric segment is a polyamino acid.

6: The micelle of claim 4, wherein said polyamino acid is polyglutamic acid.

7: The micelle of claim 1, wherein said hydrophobic moiety is selected from the group consisting of a hydrophobic small molecule, lipid, fatty acid, cholesterol, and a hydrophobic amino acid.

8: The micelle of claim 7, wherein said hydrophobic amino acid is phenylalanine.

9: The micelle of claim 1, wherein said targeting moiety is selected from the group consisting of tuftsin peptide, tuftsin receptor antibodies and antigen binding fragments thereof, folate, dextran, glycan, mannose, mannose derivatives and analogs, hyaluronic acid, GGP peptide, RGD peptide, CD11b antibody, CD14 antibody, F4/80 antibody, CX3CR1 antibody, Triggering Receptor Expressed on Myeloid Cells-1 (TREM1) antibody, TREM2 antibody, and macrophage colony stimulating-factor receptor (CD115) antibody.

10: The micelle of claim 1, wherein the inhibitor of oxidative phosphorylation is an oligomycin.

11: The micelle of claim 1, wherein the inhibitor of oxidative phosphorylation is selected from the group consisting of oligomycin A, oligomycin B, oligomycin C, oligomycin D, oligomycin E, oligomycin F, rutamycin B, 44-homooliomycin A, 44-homooligomycin B, leucinostatins, efrapeptins, resveratrol, piceatannol, (−)epigallocatechin gallate, (−)epicatechin gallate, quercetin, and genistein.

12: The micelle of claim 1, wherein said micelle is a nanoparticle.

13: The micelle of claim 12, wherein said micelle has a diameter of less than about 200 nm.

14: A composition comprising the micelle of claim 1 and at least one pharmaceutically acceptable carrier.

15: A method of treating a bacterial infection in a subject in need thereof, said method comprising administering to said subject the micelle of claim 1.

16: The method of claim 15, wherein said bacterial infection is a biofilm infection.

17: The method of claim 15, wherein said micelles are administered by injection directly to the site of infection.

18: The method of claim 15, further comprising administering at least one antibiotic and/or antibacterial drug.

19: The method of claim 15, wherein said bacterial infection is a prosthetic joint infection.


SEQUENCE LISTING (4 sequences)

SEQ ID NO: 1 - protein, 4 aa, Artificial Sequence
  tuftsin peptide
TKPR

SEQ ID NO: 2 - protein, 9 aa, Artificial Sequence
  GGP peptide
GGPNLTGRW

SEQ ID NO: 3 - protein, 3 aa, Artificial Sequence
  RGD peptide
RGD

SEQ ID NO: 4 - protein, 5 aa, Artificial Sequence
  tuftsin peptide
TKPRC